(19) United States
(12) Patent Application Publication
(10) Pub. No.: US 2026/0248407 A1
(43) Pub. Date: Aug. 27, 2026

(54) ENDOSCOPIC IMAGING AND COMPRESSION FOR ENDOSCOPIC PROCEDURES

(71) Applicant: ColoWrap, LLC., Durham, NC (US)

(72) Inventors: James HATHORN, Durham, NC (US); Anjana MOHANTY, Durham, NC (US)

(21) Appl. No.: 19/546,239

(22) Filed: Feb. 20, 2026

Related U.S. Application Data
(60) Provisional application No. 63/761,369, filed on Feb. 21, 2025.

(51) Int. Cl. A61B 5/06; A61B 1/00; A61B 1/31; A61B 5/00

(52) U.S. Cl. CPC A61B 5/062; A61B 1/00016; A61B 1/00045; A61B 1/31; A61B 5/742

(57) ABSTRACT

A method and apparatus for utilizing a model to support an endoscopic procedure are described.. The apparatus includes an input component to receive, from one or more sensors, information related to positioning of an endoscope in relation with a front portion of an endoscopy device or with a set of anatomical markers of a patient., The endoscopy device is configured for placement around an abdomen of the patient during the endoscopic procedure and configured to apply an adjustable pressure, in accordance with the model, through compression on at least part of the abdomen of the patient. The apparatus includes an output component to output, based on the information, one or more of: a flag identifying a potential loop that is predicted at the patient, a recommendation of an action predicted to avoid a first complication, or instruction to adjust an external pressure device as predicted to avoid a second complication.

### CROSS REFERENCE TO RELATED APPLICATIONS

The present Application for Patent claims priority under 35 U.S.C. § 119 to United States Provisional Patent Application No. 63/761,369 filed on Feb. 21, 2025 and entitled “Self-Learning, Autonomous Endoscopy Compression Device,” which is assigned to the assignee hereof and hereby expressly incorporated by reference herein.

### INTRODUCTION

A colonoscopy is an example of an endoscopy procedure including an examination of the large intestine or colon through the use of a colonoscope. A colonoscope is a flexible, tube-like inspection device having a camera at its end. Colonoscopies are performed for a variety of medical reasons including detection of inflamed tissue, ulcers, abnormal growths or polyps, and colorectal cancer. Colonoscopy is increasingly used as a screening tool to detect colorectal cancer.

During a colonoscopy, as an example of an endoscopy procedure, a colonoscope is inserted into a patient's rectum and then advanced to the beginning of the colon (an area known as the cecum) in order to examine the lining of the large intestine. The efficiency and accuracy of this procedure is largely dependent on the ease with which the colonoscope can be advanced. During the procedure, the colon may become over-distended or flopped in unnatural directions creating loops that hinder the advancement of the colonoscope and resulting in patient discomfort, longer examination times, and potentially inaccurate or incomplete screenings.

Currently, the difficulty in advancing the scope is addressed by the application of manual pressure by a technician to manually support the patient's colon. The application of manual pressure is time-consuming and varies depending on the particular technician’s strength, technique, endurance, and training. In order to apply differential pressure or to change the orientation of the colon within the body, the technician may roll the patient from the left side to a supine or to a prone position, which can be a difficult task with a sedated patient. The application of manual pressure and movement of the patient in order to support the patient’s colon and advance the colonoscope during the procedure places a physical toll on the technician.

### SUMMARY

In an aspect of the disclosure, a method and apparatus for assisting a user in applying pressure to the abdomen of a patient and imaging of an endoscope to ease the passage of the endoscope during procedures used to examine the bowels including colonoscopy, sigmoidoscopy, and enteroscopy. Aspects presented herein provide a representation of the positioning and/or location of the endoscope on the abdomen of the patient to assist with the advancement, withdrawal, and/or visualization of the endoscope.

The aspects presented herein exert lower abdominal pressure as well as provide information related to the positioning of the endoscope in relation to the colon including the sigmoid and transverse, and/or small bowel to assist with the advancement, withdrawal of the endoscope , and/or visualization of the lining of the colon and other anatomy as part of an endoscopy, colonoscopy, sigmoidoscopy, or enteroscopy procedure. Aspects presented herein provide improved visual tools to assist in preventing and reducing intestinal looping, eliminating the need for the application of manual pressure, improving patient safety, comfort, and satisfaction, and preventing musculoskeletal injury to endoscopy healthcare providers. Aspects may improve visualization of the anatomy and/or the insertion and withdrawal portions of the procedure. Aspects herein provide for self-adjusting pressure applications based on feedback and outputs / inferences of models, and for audio / visual indications related to endoscopic procedures based the models. Aspects herein also provide for the models to be artificial intelligence (AI) / machine learning (ML) (AI/ML) models, as well as for training / refining of such models based on a priori and current information (e.g., medical results, feedback from devices, manual inputs from medical personnel, and/or the like).

In some aspects, an apparatus utilizing a model to support an endoscopic procedure is provided. The apparatus includes an input component configured to: receive, from one or more sensors, information related to positioning of an endoscope in relation with a front portion of an endoscopy device or with a set of anatomical markers of a patient, where the endoscopy device is configured for placement around an abdomen of the patient during the endoscopic procedure and configured to apply an adjustable pressure, in accordance with the model, through compression on at least part of the abdomen of the patient, and an output component configured to output, based at least in part on the information, one or more of: a flag identifying a potential loop that is observed or predicted at the patient during the endoscopic procedure, a recommendation of an action that is predicted to avoid a first complication during the endoscopic procedure, or instruction to adjust an external pressure device, where the adjustment is predicted to avoid a second complication during the endoscopic procedure.

In some aspects, a method is provided for utilizing a model to support an endoscopic procedure. The method includes receiving, via an input component and from one or more sensors, information related to positioning of an endoscope in relation with a front portion of an endoscopy device or with a set of anatomical markers of a patient, where the endoscopy device is configured for placement around an abdomen of the patient during the endoscopic procedure and configured to apply an adjustable pressure, in accordance with the model, through compression on at least part of the abdomen of the patient, and outputting, via an output component and based at least in part on the information, one or more of: a flag identifying a loop that is observed or a potential loop that is predicted at the patient during the endoscopic procedure, a recommendation of an action that is predicted to avoid a first complication during the endoscopic procedure, or instruction to adjust an external pressure device, where the adjustment is predicted to avoid a second complication during the endoscopic procedure.

In some aspects, a non-transitory computer-readable medium storing computer executable code at a device is provided, which utilizing a model to support an endoscopic procedure. The code when executed by a processor / processing circuitry causes the processor processing circuitry to: receive, via an input component and from one or more sensors, information related to positioning of an endoscope in relation with a front portion of an endoscopy device or with a set of anatomical markers of a patient, where the endoscopy device is configured for placement around an abdomen of the patient during the endoscopic procedure and configured to apply an adjustable pressure, in accordance with the model, through compression on at least part of the abdomen of the patient, and output, via an output component and based at least in part on the information, one or more of: a flag identifying an observed loop or predicting a potential loop at the patient during the endoscopic procedure, a recommendation of an action that is predicted to avoid a first complication during the endoscopic procedure, or instruction to adjust an external pressure device, where the adjustment is predicted to avoid a second complication during the endoscopic procedure.

Additional advantages and novel features of aspects of the present invention will be set forth in part in the description that follows, and in part will become more apparent to those skilled in the art upon examination of the following or upon learning by practice thereof.

### BRIEF DESCRIPTION OF THE DRAWINGS

FIG. 1A is a schematic view of a colon with an endoscope (also known as a colonoscope for colonoscopy procedures) partially inserted therein.

FIG. 1B is a schematic view of a colon in which a sigmoid loop has developed due to an attempt to advance the endoscope against an unsupported colon wall.

FIG. 1C is a schematic view of a colon showing the application of manual pressure to the colon to facilitate insertion of an endoscope.

FIG. 2A is a front view of an endoscopy device or compression device, in accordance with aspects presented herein.

FIG. 2B is a rear view of an endoscopy device or compression device, in accordance with aspects presented herein.

FIG. 3A is a front view of an endoscopy device or compression apparatus, in accordance with aspects presented herein.

FIG. 3B is a rear view of an endoscopy device or compression apparatus, in accordance with aspects presented herein.

FIG. 4A is a front view of an endoscopy device or compression device, in accordance with aspects presented herein.

FIG. 4B is a rear view of an endoscopy device or compression device, in accordance with aspects presented herein.

FIG. 5 is a diagram of an imaging system, in accordance with aspects presented herein.

FIG. 6 is a diagram of an imaging system, in accordance with aspects presented herein.

FIG. 7 is a diagram of a visual representation of a scope during an endoscopic procedure, in accordance with aspects presented herein.

FIG. 8 is a diagram of a visual representation of a scope during an endoscopic procedure, in accordance with aspects presented herein.

FIG. 9A is a diagram of a visual representation of a scope during an endoscopic procedure, in accordance with aspects presented herein.

FIG. 9B is a diagram of a visual representation of a scope during an endoscopic procedure, in accordance with aspects presented herein.

FIG. 10 is a diagram of an LED array for visual indications during an endoscopic procedure, in accordance with aspects presented herein.

FIG. 11 is a diagram of a computer system on which the disclosed system and method can be implemented, in accordance with various aspects of presented herein.

FIG. 12 illustrates example aspects of model implementations and training, in accordance with aspects presented herein.

FIG. 13 is a flowchart of a method for support of an endoscopic procedure.

FIG. 14 illustrates example aspects of model implementation, training, and inference in accordance with aspects presented herein.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

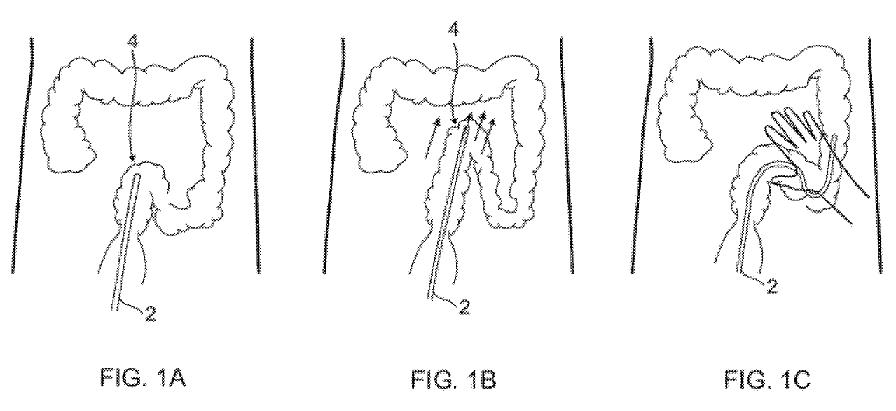

FIGS. 1A-1C, illustrate a sequence of steps of a colonoscopy, as one example of an endoscopy procedure. In FIG. 1A, a colonoscope 2 is inserted into the patient’s rectum and advanced forward through the length of the colon. As the operator passes the colonoscope through the sigmoid region of the colon 4, the colonoscope may become impinged and cause distention and looping of the anatomy, as shown in FIG. 1B. The distention causes discomfort to the patient and increases the time required for the colonoscopy. In order to reduce the distended or looped area, a technician may apply manual pressure to the abdomen of the patient. Among other examples, the technician may be a nurse, assistant, or other staff member. For example, the pressure may be applied by a nurse or surgical assistant as shown in FIG. 1C.

The application of manual pressure is time-consuming and places a physical toll on the technician. The effectiveness of the manual pressure varies depending on the particular technician’s strength, technique, endurance, and training. In order to apply differential pressure and change the orientation of the colon within the body, the technician may roll the patient from the left side to a supine or to a prone position, which can be a difficult task with a sedated patient. The application of manual pressure and movement of the patient in order to support the patient’s colon and advance the colonoscope during the procedure may lead to injury of the patient or of the technician.

Many patients undergo colonoscopy while placed in the left lateral decubitus position on the stretcher or operating table. Additional information about the use of such manual pressure can be found in Prechel JA, Hucke R. Safe and effective abdominal pressure during colonoscopy: forearm versus open hand technique. Gastroenterol Nurs 2009;32:27-30; quiz 31-2, the entire contents of which are incorporated herein by reference. In applying manual pressure, the technician may reach over the patient from the opposite side of the table and to deploy pressure by placing their hands against the patient’s sigmoid colon and then leaning backwards, using their bodyweight for leverage to exert force. While these methods are generally effective at generating pressure, they have also been identified as a causative factor for the high rate of work-related injuries among endoscopy nurses and staff. Physicians performing colonoscopy suffer work-related musculoskeletal injury at a particularly high-rate as well. The most frequent site of physician injury is the right upper extremity which experiences peak torque forces when while operators are attempting to advance the scope through (a looping) sigmoid colon. Additional details can be found in Spanarkel M, Hathorn JP. Looping During Colonoscopy: A Major, Implied Cause of Injury Among Endoscopy Healthcare Providers and a Proposed Solution, 2013, the entire contents of which are incorporated herein by reference.

Aspects presented herein assist the technician to more effectively provide targeted compression to a patient’s abdomen during an endoscopy procedure. Aspects include visual components that are more intuitive for the technician to identify the location or position of the endoscope within the patient’s abdomen during an endoscopy procedure. By assisting the technician in more readily, or more accurately, identifying the position of the endoscope, the technician can more effectively apply targeted pressure during the procedure. The pressure may be applied manually or may be applied through adjustment of a compression device. In some aspects, a visual identification of the endoscope position may be provided at the patient’s abdomen, such as at an LED array that is placed at the patient’s abdomen. In some aspects, the visual identification may be provided at a display screen that shows the location of the endoscope relative to anatomical landmarks to enable the technician to more readily interpret the visual display and apply pressure at a corresponding location on the patient.

Aspects described herein may similarly be applied for other endoscopic procedures such as, but not limited to, sigmoidoscopy and retrograde enteroscopy procedures. Sigmoidoscopy is an examination of only the lower part of the colon, from the anus to the descending colon. An endoscope is inserted into the lower part of the colon. Enteroscopy is an examination of the small bowel. During retrograde enteroscopy, an endoscope is inserted in the anus and passed through the colon and the cecum and into the small bowel. Successfully navigating the loop-prone sigmoid region is necessary to complete both sigmoidoscopy and retrograde enteroscopy and thus aspects described herein can be used to help facilitate colonoscopy, sigmoidoscopy, retrograde enteroscopy, and other endoscopic procedures.

Each of the techniques can be used via an antegrade approach or a retrograde approach. In an antegrade approach, the scope is introduced through the patient’s mouth and is advanced through the stomach in order to visualize the small intestine. In a retrograde approach, the scope is introduced through the rectum and through the colon and cecum in order to visualize the small intestine.

As described herein, one or more compression apparatuses can be applied at / around a patient enabling compression to be applied and/or adjusted at various points of the different procedures, e.g., as the scope is iteratively advanced deeper into the small bowel. The different levels and mechanisms of compression, presence/absence of compression, and/or targeted areas of compression may assist with the advancement of the scope, withdrawal of the scope, and/or visualization of the small bowel, colon, etc.

In order to apply effective compression and/or targeted pressure, it would be helpful for a user to know a location of the endoscope during the procedure, e.g., during insertion, visualization, and/or withdrawal. Aspects presented herein provide tools that assist the user in visualizing a location of the endoscope during an endoscopy procedure.

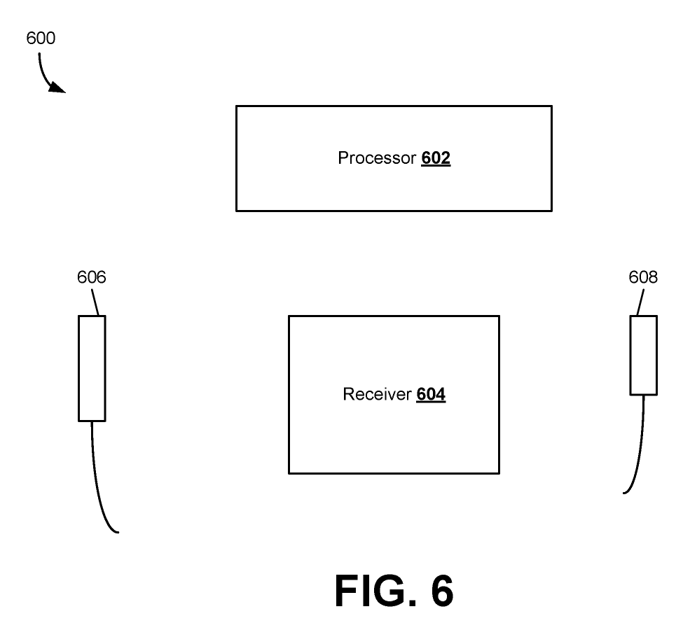

In some instances, as shown in diagram 600 of FIG. 6, a magnetic endoscopic imaging system that includes a scope 608 and controller 606, that may be utilized to assist in the visualization of the scope during the endoscopic procedure. The magnetic endoscopic imaging system may provide a representation of the position and/or configuration of the scope within the small or large bowel. The scope 608 may include electromagnetic coils along the length of the scope 608 that generate a pulsed low-intensity magnetic field that is detected by a receiver 604, where the receiver, scope, and coils interface with a processor 602. The low-intensity magnetic field is utilized to generate a representation of the position and configuration of the scope on a monitor. For example, the three-dimensional representation may be displayed on a monitor / display which may provide instant feedback as the scope is advanced during the procedure.

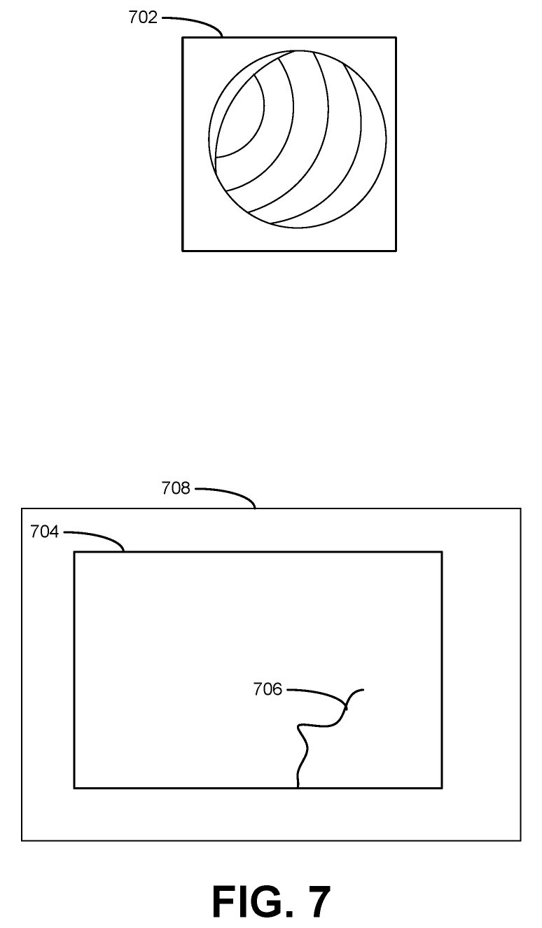

Although image orientation may be toggled or adjusted in conjunction with adjustments in patient body position (e.g. left lateral vs. supine vs. right lateral vs. prone) the magnetic endoscopic imaging system conveys limited information of the position of the scope. Aspects presented herein provide a system that improves user identification of scope location by rendering the image of scope shape and orientation onto the patient’s body and/or convey the scope image relative to a set of common anatomical landmarks (also “marks” or “markers”). For example, the representation of the scope 706 location displayed on the monitor, as shown in FIG. 7, is shown in free space (e.g., 704) at a monitor 708 and does not include any reference points which may indicate the positioning of the scope 706 within the bowel 702 of a patient, which may limit the utility of the information being conveyed.

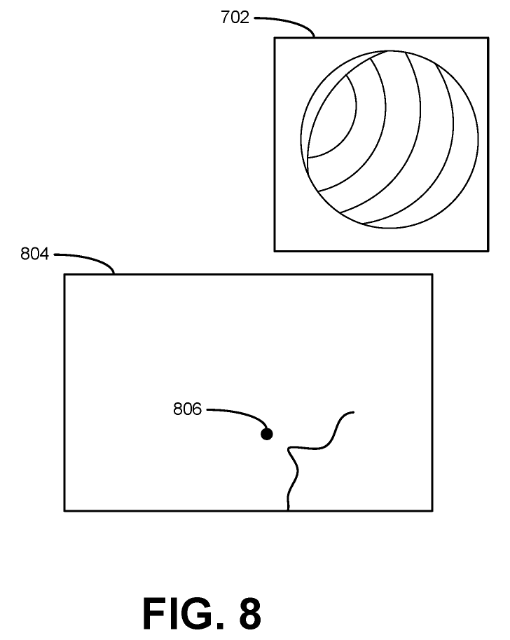

In some instances, as shown for example in FIG. 8, the magnetic endoscopic imaging system may provide an indication 806 on the display 804 where abdominal pressure intervention may be exerted to assist in the advancement of the scope. The indication 806 may correspond to the location of a device that is held by a user at the exterior of the abdomen of the patient. The indication 806 can be displayed on the display with respect to the three-dimensional representation of the scope. However, the utility of such indication to direct any type of abdominal pressure intervention may be limited or non-intuitive relative to the patient. It can be difficult for a practitioner to use the indication on the monitor to determine where the abdominal pressure intervention should be applied onto the patient’s body.

Aspects presented herein provide an apparatus configured to convey a representation of the scope location with an image relative to a body outline and/or relative to common anatomical landmarks of the patient. Examples of such common anatomical landmarks include a marker that can be identified externally, such as a marker placed at the patient, or a body part such as the bones of the hip(s), pelvis, ribcage, spine, and/or the like. At least one advantage is that this would provide an enhanced representation of the scope with relation to the patient’s body for scope location and looping location specific to the position of the actual patient. In some instances, the scope image may be shown relative to known anatomical features of the patient (e.g., skeletal structure, etc.) or relative to an outline of the patient’s body.

Aspects presented herein may further provide an apparatus configured to provide the location of the scope and/or an indication of a location for abdominal pressure relative to the body outline and/or relative to common anatomical landmarks would provide an enhanced indication as to where to apply abdominal pressure. For example, the scope image and the indication of a location for abdominal pressure may be shown relative to known anatomical features of the patient (e.g., skeletal structure, etc.) or relative to an outline of the patient’s body. In another example, the apparatus may be configured to show a representation of the shape and/or location of the scope onto the patient’s body.

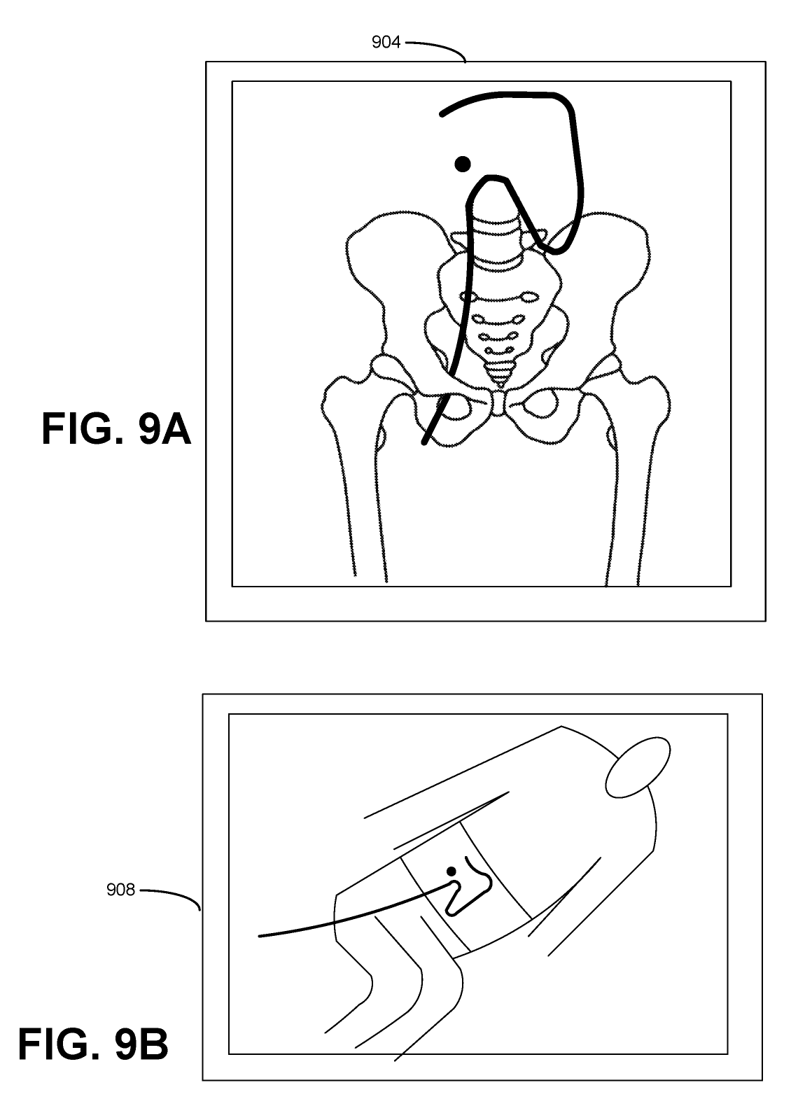

In some aspects, a colonoscopy device may be worn by or placed on a patient, where the colonoscopy device comprises one or more embedded coils and/or sensors that may alsobe identified by the receiver of the magnetic endoscopic imaging system. In some examples, the device may also be used as a compression device. The aspects to provide a visual indication of a location of an endoscope may also be incorporated into a non-compressive device that surrounds, or is placed over, at least a portion of the abdomen of the patient. The device may be referred to by any of various names, e.g., a compression device, an external device, an endoscopy device, an endoscopy visualization device, endoscopy visualization tool, an endoscopy landmark device, or an endoscopy marker device, among other examples. The one or more embedded coils and/or sensors may be configured to be aligned with specific areas on the device that may be aligned with specific anatomical locations of the patient. For example, the device may be placed with specific areas aligned with the patient’s hips or a specific area aligned with the patient’s navel. The device may be configured to remain in a fixed location at the patient during the procedure so that the information received from the sensors in the device enable a consistent determination of endoscope position relative to the device and therefore relative to the anatomical landmarks of the patient. This may allow the position and/or configuration of the scope to be conveyed (e.g., visually displayed) relative to the anatomical locations of the patient, which would enable an enhanced representation of the scope in relation with the patient’s body, as shown for example in FIGS. 9A or 9B. For example, FIG. 9A illustrates an example display 904 that shows a position of the endoscope relative to an illustration of anatomical features. FIG. 9B illustrates an example of a display 908 that shows a position of the endoscope relative to an illustration of an outline of a patient.

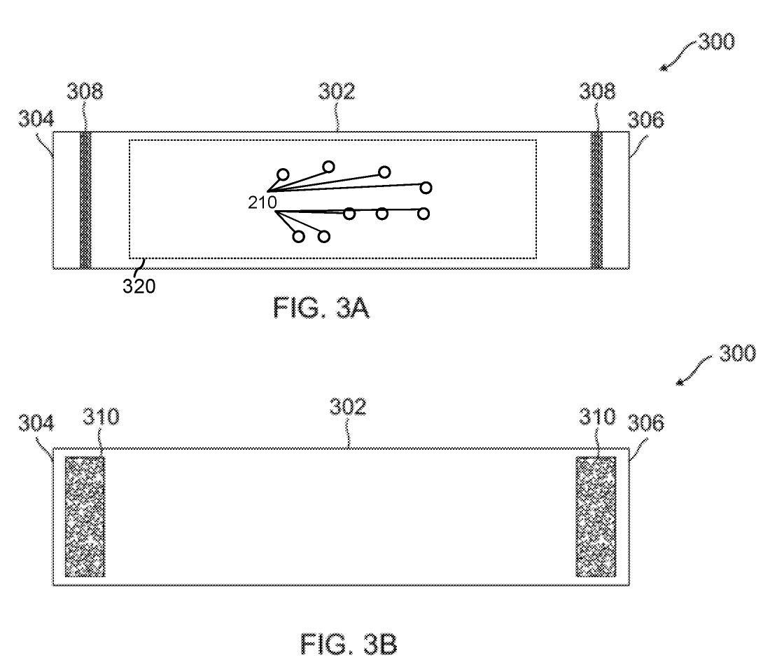

In some aspects, the endoscopy device (e.g., the compression apparatus 300 in FIG. 3A or 3B) may comprise a wrap or a sleeve configured to surround at least a portion of an abdomen of the patient. In some aspects, the endoscopy device may include a layer that is placed over the patient’s abdomen. The layer may include a rectangular layer, a rounded layer, a shaped layer that is wider at the hips and narrower toward the bottom of the abdomen, the layer may be shaped to cover a substantial portion of the abdomen. The device may be placed on or secured to the abdomen of the patient. In some aspects, at least a portion of the layer may adhere to the patient’s abdomen. The layer may include the one or more sensors to detect a magnet at the endoscope and/or may include LEDs to provide a visual representation of the endoscope location within the patient. The device may comprise one or more sensors configured to provide information related to the position of the scope in relation with at least the front portion of the device. In some aspects, the device may be configured to apply pressure (e.g., including an adjustable pressure via self-adjustment based on output(s) / a model, as described herein) on at least part of the abdomen of the patient during an endoscopy procedure. In some aspects, the one or more sensors may detect one or more magnets of the scope as the scope traverses the bowel during the endoscopic procedure. In some aspects, the one or more sensors may provide the information related to the positioning of the scope in relation with anatomical landmarks of the patient. The one or more sensors may provide the information related to the positioning of the scope in relation with anatomical landmarks of the patient based at least on the one or more sensors being aligned with the anatomical landmarks of the patient. In some aspects, the positioning of the scope may be detected in relation to the one or more sensors.

In some aspects, a pressure indication may be provided in relation with the anatomical landmarks of the patient. For example, the pressure indication may indicate a location for external pressure on the abdomen of the patient to assist with the scope traversing the bowel of the patient. In aspects, the external pressure may include an adjustable pressure via self-adjustment, as described herein.

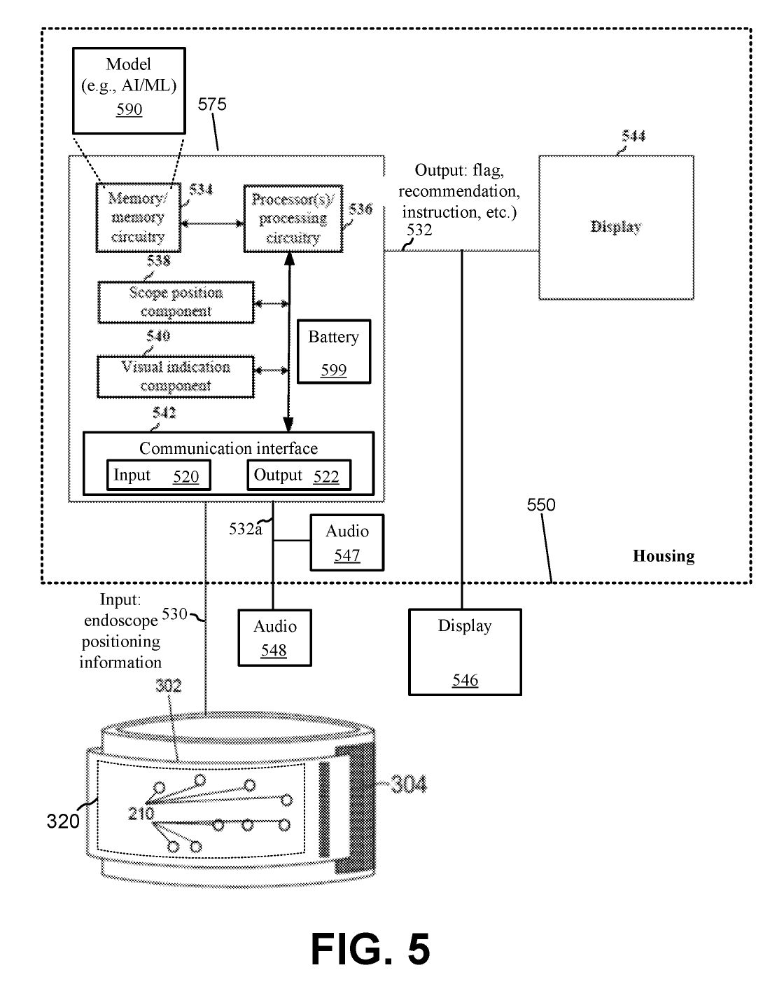

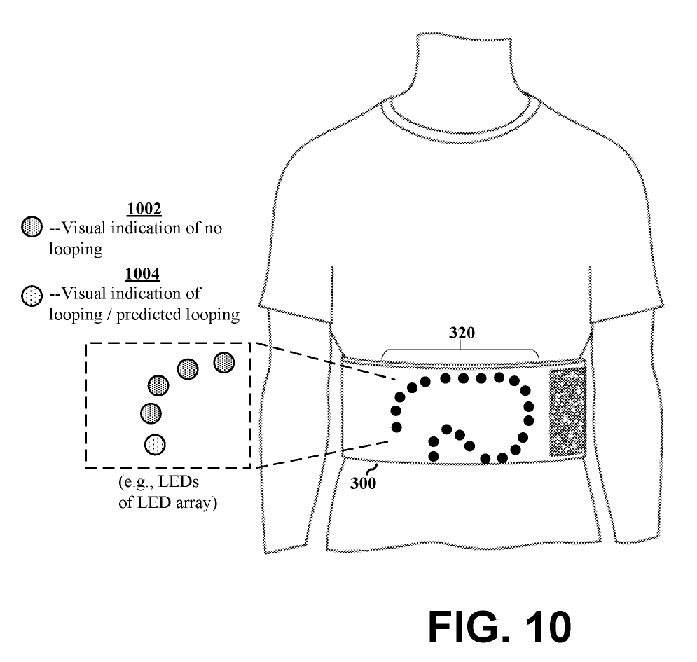

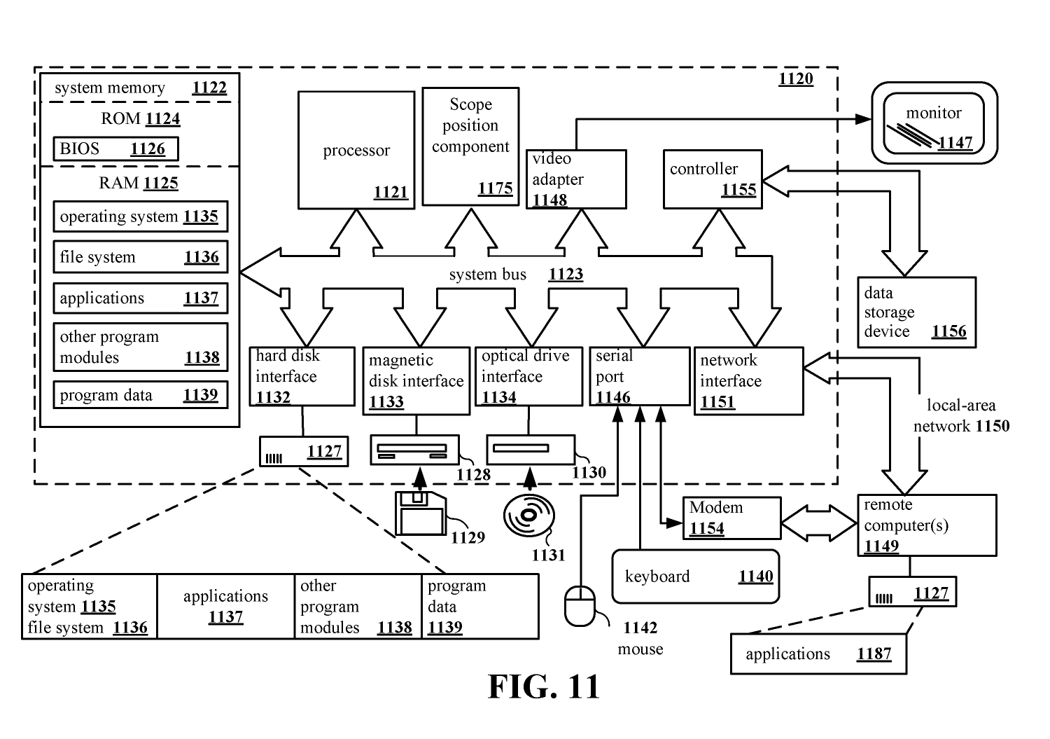

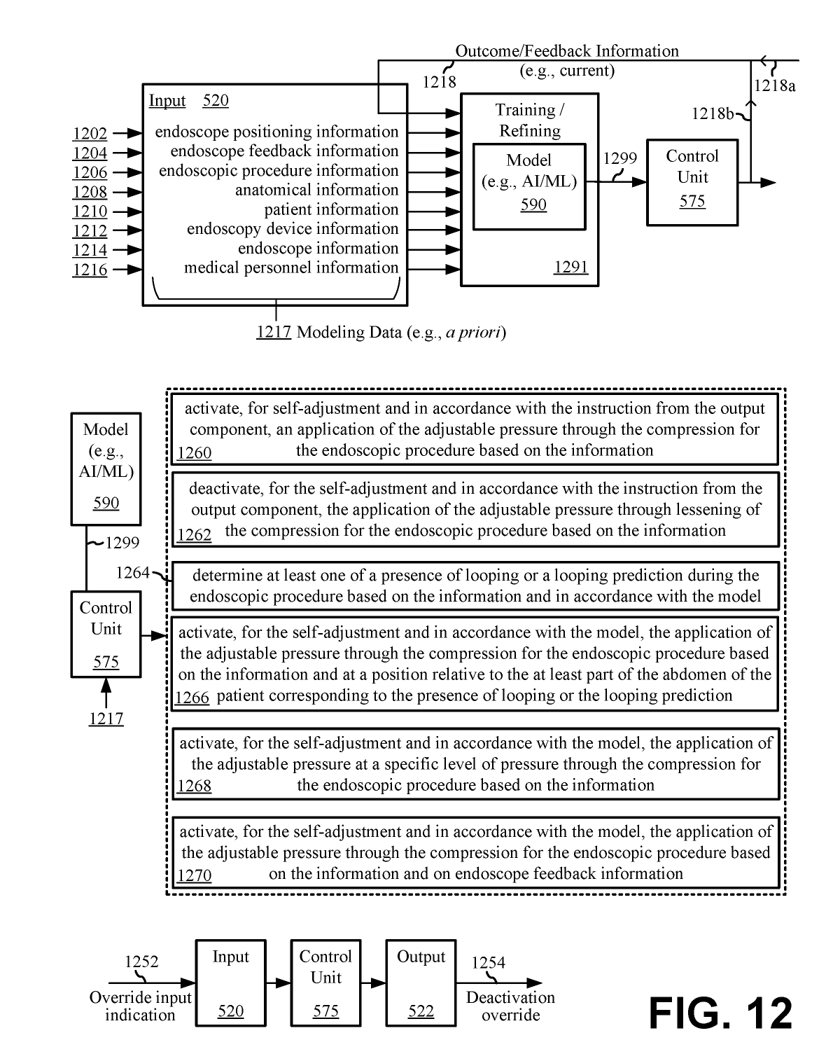

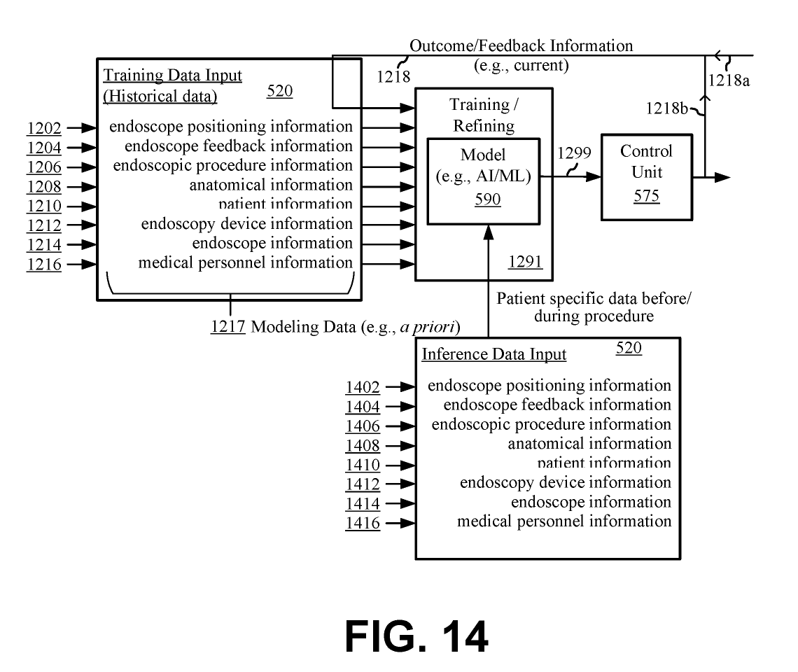

In some aspects, as shown for example in FIG. 10, the device may comprise a light emitting diode (LED) array. The aspects described in connection with FIG. 10 may be provided as an alternative to, or an addition to, the display aspects described in connection with FIGS. 9A and 9B. One or more LEDs of the LED array (e.g., the LED array 320 of the compression apparatus 300 in FIG. 3A) may be activated (to emit light) to correspond with the position and a shape of the endoscope in response to the endoscope being proximate to the one or more LEDs of the LED array. For example, the LEDs may be activated by the magnetic field from the scope or may be activated by a signal emitted by the scope. In such instances, the sleeve may comprise a control unit comprising at least one processor coupled to at least one receiver (e.g., an input component 520) and at least one transmitter (an output component 522). Example aspects of a control unit are illustrated in FIG. 5. The control unit may receive the signal emitted from the scope, as received by the one or more sensors 210 via the input component 520 (e.g., which may be capable of wired / wireless communications), to detect, identify, or determine the positioning of the scope. FIG. 5 illustrates an example in which the control unit 575 may include memory or memory circuitry 534 and one or more processors or processing circuitry 536 (e.g., a processor, at least one processor, and/or the like) configured to cause a visual indication of a position of an endoscope relative to a patient during an endoscopy procedure as described herein. In some aspects, the control unit 575 may be configured to cause the position of the endoscope to be displayed via the output component 522 at the display 544 / the display 546 as described in connection with FIG. 9A and 9B. In some aspects, the control unit 575 may be configured to cause the position of the endoscope to be displayed via the output component 522 in an LED array on the device at the abdomen of the patient, e.g., as described in connection with FIG. 10. In some aspects, the LED array may be part of a device, wrap, or sleeve that surrounds the patient, e.g., to hold the LED array in position at the abdomen of the patient. In some aspects, the control unit 575 may be provided at, comprised with, and/or removably attachable to the device (e.g., 302) that is configured to be positioned at / around the abdomen of the patient. The control unit 575 may provide a signal to the display 544 / the display 546 via a wired or wireless coupling via the communication interface 542 (e.g., via the output component 522). In some aspects, the control unit 575 may provide an audio signal to an audio component 547 or an audio component 548 via a wired or wireless coupling via the communication interface 542 (e.g., via the output component 522 and/or connection 532a). The audio signal may comprise an indication that corresponds to, or is in lieu of, the signal for the display 544 / the display 546, such a voice indication(s), an audible tone(s), and/or the like. Although the display is shown as being within a housing 550, in some examples, in other examples, the display 544 may be outside of a housing that houses one or more other components of the control unit (e.g., as shown for the display 546). In some aspects, the control unit 575 may be in a device that is separate from the device (e.g., 302) that is positioned at / around the patient. In such aspects, the control unit 575 may receive sensor information from and/or transmit control information (to control lights within the LED array) to the components provided at the device 302 via a wired or wireless coupling via the communication interface 542. In some aspects, the control unit 575 may be comprised in a housing 550 with the display 544. The control unit 575 may further include a battery 599 or power source. The control unit 575 may include a scope position component 538 that is configured to receive the sensor information via the communication interface 542 (e.g., via the input component 520) and translate the sensor information to a representation of the location of the endoscope within the patient, e.g., whether for display at a monitor and/or for display via an LED array 320 at the device 302. In some aspects, the scope position component 538 may receive a signal based on the position of the endoscope and/or based on a position of the device 302. The signal may be a reflected signal, e.g., based on a reflection of a signal transmitted by the scope position component 538. The signal may be a detection or reception of a pulsed signal provided by a component at the endoscope and/or the device 302. As an example, the endoscope may include an electro-magnetic coil component that can be detected (such as through pulses) by the scope position component 538. Similarly, the device 302 may include electro-magnetic component(s) that can be detected (such as through reception of pulses) by the scope position component 538. Thus, the endoscope and/or the device 302 may include a component that provides a signal to be received and interpreted by the scope position component 538, or the scope position component 538 may transmit a signal and may perform a measurement based on its own signal (e.g., such as a reflection) to determine information about the endoscope and/or device 302. By detecting the position of both the endoscope and the device 302, the scope position component may more accurately determine the location of the endoscope for display (e.g., either for display at the device 302 itself) and/or at a remote display using more detailed anatomical information. For example, the device 302 may be placed with a specific location relative to anatomical features of the patient, and the location of the device 302 gives a landmark or reference point for a determination of the scope position within the anatomy of the patient. In some aspects, the scope position component 538 may be included in the device 302, and may detect the location of the endoscope without a further detection of the location of the device 302. The control unit 575 may further include a visual indication component 540 that is configured to provide (e.g., via the output component 522) signals to the display 544 / the display 546 and/or LED array 320 to visually indicate the position of the endoscope relative to the anatomy of the patient. The memory or memory circuitry 534 may include / store a model 590 that is executed / executable by the processor(s) / processing circuitry 536 of the control unit 575, in aspects. The model 590 may be an AI model, an ML model, an AI/ML model, and/or the like. Aspects described with respect to FIG. 5, and elsewhere herein, such as but not limited to applications of adjustable pressure (e.g., via self-adjustment), may be implemented in accordance with / based on the model 590. In one example, based on input 530 (e.g., information related to positioning of an endoscope in relation with a front portion of an endoscopy device or with a set of anatomical markers of a patient), received via the input component 520 and from the one or more sensors 210, the control unit 575 may be configured, based on the model 590, to cause output 532 (e.g., a flag identifying an observed loop or predicting a potential loop at the patient during the endoscopic procedure, a recommendation of an action that is predicted to avoid a first complication (e.g., looping) during the endoscopic procedure, instruction to adjust an external pressure device (e.g., for an adjustable pressure via self-adjustment), where the adjustment is predicted to avoid a second complication (e.g., looping) during the endoscopic procedure, and/or the like) to be provided to the display 544 / the display 546. FIG. 11 illustrates an example of a processing system that includes additional aspects and components that may be included in or coupled to the control unit 575. FIG. 12 illustrates example aspects of model 590 implementations and training, in accordance with aspects presented herein. FIG. 14 illustrates example aspects of input of historical data for training the model 590 and the input of inference data for a particular patient, prior to or during a procedure, in order to obtain an inference output from the trained model 590.

The positioning of the scope may be represented by the LED array, where the LED array is on or within the front portion of the sleeve. In some aspects, the LED array may be embedded within the front portion of the sleeve or compression device, such that one or more LEDs of the LED array may be activated in response to the scope traversing the bowel. In some aspects, the LED array may be embedded within one or more compression apparatuses or the sleeve. The device may extend over the abdomen of the patient and may be configured to apply a pressure upon at least a portion of the abdomen of the patient. For example, the device may include any of the aspects described in connection with U.S. Patent No. 11,701,286 titled “Endoscopy Band with Sigmoid Support Apparatus” that issued on July 18, 2023, the entire contents of which are incorporated herein by reference. The one or more compression apparatuses extending over the abdomen of the patient may allow for the LEDs of the LED array to be activated in response to the scope traversing the bowel.

In some aspects, the LED array may provide a visual indication 1002 to indicate an absence of looping and/or a visual indication 1004 to indicate that looping is likely to be occurring and/or to recommend that compression be applied to assist with movement of the endoscope. For example, the LED may illuminate with a first color to show the position of the endoscope when there is no looping, and may illuminate with a second color to indicate potential looping and/or to indicate a potential location for compression to assist with movement of the endoscope within the patient. The control unit, or another component of the device, may identify and/or predict looping based on the sensor information received from the one or more sensors, for example.

In some aspects, the control unit may be configured to transmit a positioning signal that indicates the positioning of the scope based on the signal from the scope. The receiver of the magnetic endoscopic imaging system may receive the positioning signal from the transmitter of the control unit to generate the three-dimensional image of the scope in relation with anatomical landmarks of the patient.

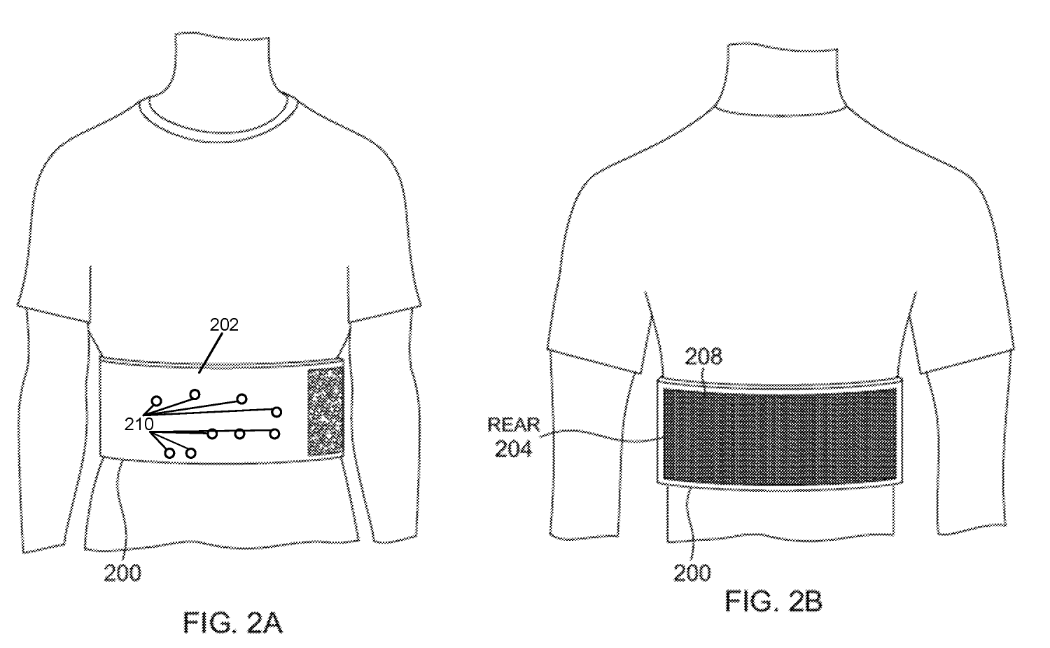

FIGS. 2A and 2B illustrate an example sleeve device. The sleeve 200 comprises a front portion 202, a rear portion 204, one or more anchor regions 208, and one or more sensors 210 on (e.g., as part of, attached to, etc.; generally “comprising”) at least the front portion 202 or the rear portion 204. Although referred to as a sleeve, the device may include a band that is configured to be fastened at / around the abdomen of the patient. The sleeve 200 is configured to surround at least a portion of the abdomen of the patient during endoscopic procedures (e.g., at least partially circumscribe the body of the patient while covering the abdomen, etc.). In some aspects, the sleeve 200 may act as a modular base that may be worn by patients, or placed under patients, in preparation of and/or during an endoscopy procedure that may require application of external and/or localized pressure to assist or allow the endoscope, or similar instrument, to advance the instrument during the procedure. The sleeve 200 may be configured as a modular base that may be worn by patients that may act as an anchoring base for one or more compression apparatuses that may deliver the external and/or localized pressure as desired. The sleeve may receive a variety of compression apparatuses, bands or attachments that may have different purposes. In some aspects, the sleeve may include a unitary section that encircles the abdomen of the patient. In some aspects, the sleeve may be a portion of a more comprehensive endoscopy garment.

The front portion 202 of the sleeve, when worn by the patient, may cover an abdominal region of the patient, while the rear portion 204 may cover a lower back region of the patient. The front portion 202 may be comprised of a first material that may have elastic properties. The first material of the front portion 202 may expand to accommodate various abdomen sizes of different patients to allow the sleeve to be utilized for patients of different body sizes. The rear portion 204 may be comprised of a second material that has less elasticity than that of the first material. In some aspects, the second material may be comprised of a semi-rigid or reinforced material. In some aspects, the second material may be comprised of one or more materials having different elasticities. For example, the second material may have a first elasticity along a rear central region of the rear portion 204, while the second material may have a second elasticity along the outer ends of the rear portion 204 that are proximate the front portion 202 of the sleeve. The first elasticity may be less elastic than the second elasticity, such that the second material having the first elasticity is maintained substantially aligned with a spine of the patient. The rear portion comprising the second material may maintain or align the rear portion 204 of the sleeve onto a back region of the patient. The rear portion 204 comprising the second material may be configured to provide a support structure to assist the one or more compression apparatuses to apply the pressure during the procedure. In some aspects, the rear portion 204 may counter pressure applied by the one or more compression apparatuses to maintain or align the rear portion 204 of the sleeve with the back region of the patient.

The one or more sensors 210 may be configured to be aligned with anatomical landmarks of the patient. The one or more sensors may be configured to obtain and/or provide information / indications related to the positioning of the scope in relation with at least the front portion 202 of the sleeve. In some aspects, the one or more sensors may obtain information about / detect one or more magnets of the scope in response to the scope traversing the bowel during the endoscopic procedure. In some aspects, the one or more sensors may detect the positioning of the scope in relation with anatomical landmarks of the patient. The one or more sensors may detect the positioning of the scope in relation with anatomical landmarks of the patient based at least on the one or more sensors being aligned with the anatomical landmarks of the patient, the one or more magnets of the scope, and/or one or more magnetic field pulses emitted from the scope.

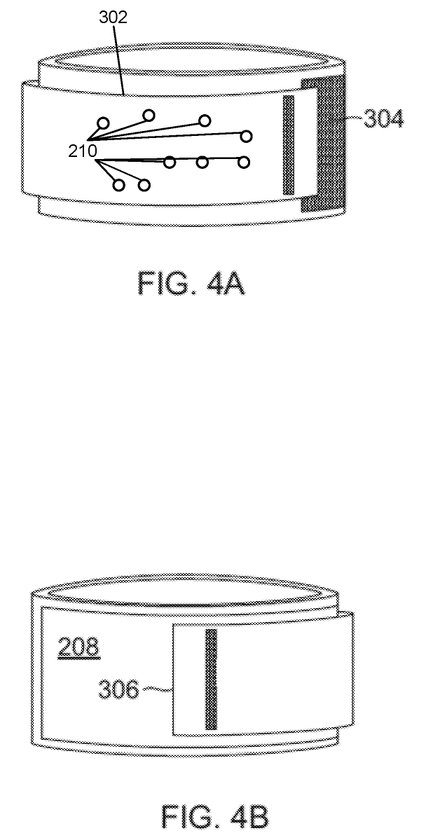

FIGS. 3A and 3B illustrate an example of an endoscopy device to be placed at the patient’s abdomen, which may be configured as a compression apparatus 300. The compression apparatus 300 comprises a first end 304, a body of the device 302, and a second end 306. The first end 304 may be configured to be removably coupled to the front portion of the sleeve, as shown for example in FIG. 4A. The second end 306 may be configured to be removably coupled to the rear portion of the sleeve, such that the compression apparatus 300 may extend over the abdomen of the patient and apply the pressure upon at least a portion of the abdomen of the patient, as shown for example in FIGS. 4A and 4B.

The first end 304 of the compression apparatus 300 may comprise a fastener 310 such that the first end may couple to the rear portion 204 of the sleeve 200 (e.g., in FIG. 2B). For example, the fastener 310 may comprise a plurality of hooks while the rear portion of the sleeve comprises a plurality of loops, such that the plurality of hooks of the fastener 310 of the first end 304 may couple with the sleeve. In some aspects, the fastener 310 may comprise a plurality of loops while the front portion of the sleeve comprises a plurality of hooks, such that the first end may couple with the sleeve. The fastener 310 may comprise many different fastening devices and is not intended to be limited to a hook or loop configuration and may include snaps, adhesive, buttons, ties, or other fasteners. The fastener 310 is configured to correspond with the front portion 202 of the sleeve 200 such that the first end 304 may be removably coupled to the sleeve.

The second end 306 of the compression apparatus 300 may also comprise a fastener 310 configured in a similar manner as fastener 310 of the first end 304. The second end 306 of the compression apparatus 300 may be removably coupled to the rear portion of the sleeve. In some aspects, the second end of the compression apparatus may be removably coupled to at least the rear portion of the sleeve. The body of the device 302 of the compression apparatus 300 may extend over a portion of the abdomen of the patient and over part of the front portion of the sleeve, such that when the first end 304 is coupled to the front portion and the second end 306 is coupled to the rear portion while the body (e.g., 302) applies pressure on the portion of the abdomen of the patient. In some aspects, the compression apparatus 300 may comprise the one or more sensors 210, and may operate in a similar fashion as described above.

In some aspects, the one or more sensors 210 may comprise one or more LEDs of an LED array 320, such that the LED array 320 is comprised within the compression apparatus 300. In such aspects, one or more LEDs of the LED array 320 may be activated to correspond with the positioning and a shape of the scope in response to the scope being proximate to the one or more LEDs of the LED array 320. For example, the LEDs may be activated by the magnetic field from the scope or may be activated by a signal emitted by the scope. In such instances, the compression apparatus 300 may communicate with a control unit comprising at least one processor coupled to at least one receiver and at least one transmitter. The control unit may be configured receive a signal emitted from the scope as the scope traverses the bowel to detect the positioning of the scope. The positioning of the scope may be represented by the LED array 320, where the LED array 320 is arranged onto the front portion of the sleeve by the compression apparatus 300. In some aspects, the LED array 320 may be embedded within one or more compression apparatuses. The one or more compression apparatuses may be removably coupled to the sleeve and may comprise a first end and a body. The one or more compression apparatuses may extend over the abdomen of the patient to apply a pressure upon at least a portion of the abdomen of the patient. The one or more compression apparatuses extending over the abdomen of the patient may allow for the LEDs of the LED array 320 to be activated in response to the scope traversing the bowel.

In some aspects, the surface of the first anchor region may comprise a fastener that corresponds with a fastener on the first end of the compression apparatus, in order to removably couple the compression apparatus to the first anchor region. In some aspects, the first anchor region may comprise a plurality of anchor regions that collectively extend along at least a portion of the sleeve.

The first end 304 of the compression apparatus 300 may comprise the fastener 310 such that the first end may couple to the at least one attachment extension 206 of the sleeve 200. For example, the fastener 310 may comprise a plurality of hooks while the at least one attachment extension 206 comprises a plurality of loops, such that the plurality of hooks of the fastener 310 of the first end 304 may couple with the at least one attachment extension 206. In some aspects, the fastener 310 may comprise a plurality of loops while the at least one attachment extension 206 comprises a plurality of hooks, such that the first end may couple with the at least one attachment extension. The fastener 310 may comprise many different fastening devices and is not intended to be limited to a hook or loop configuration. The fastener 310 is configured to correspond with the attachment extension 206 of the sleeve 200 such that the first end 304 may be removably coupled to the attachment extension 206.

The second end 306 of the compression apparatus 300 may also comprise a fastener 310 configured in a similar manner as fastener 310 of the first end 304. The second end 306 of the compression apparatus 300 may be removably coupled to the one or more anchor regions 208. The body (e.g., 302) of the compression apparatus 300 may extend over a portion of the abdomen of the patient, such that when the first end 304 is coupled to the attachment extension 206 and the second end 306 is coupled to the one or more anchor regions 208, the body (e.g., 302) applies pressure on the portion of the abdomen of the patient.

In some aspects, the first end 304 or the second end 306 of the compression apparatus 300 may each comprise a handle 308. The handle 308 may be on the band opposite the fastener 310. The handle 308 at the first end 304 may be configured to assist with the placement or positioning of the first end 304 with the attachment extension 206. The handle 308 at the second end 306 may be configured to assist with the placement or positioning of the second end 306 with the anchor region 208.

Improving patient comfort and reducing complications, both during and following endoscopic procedures is very important. Aspects presented herein reduce patient discomfort and complications by helping to prevent and reduce sigmoid looping, which can be a primary cause of patient pain and discomfort.

To additionally enhance patient comfort, certain aspects may be designed to be single-use, and to remain fastened in place on the patient during the procedure and/or following the procedure. For example, maintaining the compression applied by the device during the withdrawal phase of the procedure and while imaging is performed may help improve the detection of adenoma. The device may be maintained on the patient to reduce the common post-procedure complications of bloating and abdominal pain caused by bloating. Otherwise known as gaseous distention, bloating occurs following endoscopy procedures because physicians often use compressed air or carbon dioxide to insufflate parts of the bowel that are difficult to see and examine. The gas opens up the area to allow for a more complete visualization, enhancing the efficacy of the procedure. However, the gas also remains in the patient until it is either absorbed or expelled. Expulsion is the primary gas removal mechanism as absorption is a very inefficient process. Gaseous distention is a primary post-procedure complication and a frequent complaint from patients. However, when the wrap described herein remains in place after the procedure, the lower abdominal compression generated by the device allows the bowel to more rapidly evacuate trapped by directing excess gas towards the rectum. As a result, the severity and duration of post-procedure bloating and associated abdominal pain may be reduced.

FIG. 11 is a block diagram illustrating a general-purpose computer system 1120 on which aspects of systems and methods for providing an improved display of a location of an endoscope within a patient during an endoscopy procedure, e.g., as described in connection with any of FIGS. 1-5, 9A, 9B, and 10 may be implemented in accordance with an example aspect.

As shown, the computer system 1120 (which may be a component of a personal computer or a server) includes a central processing unit 1121, a system memory 1122, and a system bus 1123 connecting the various system components, including the memory associated with the central processing unit 1121. As will be appreciated by those of ordinary skill in the art, the system bus 1123 may comprise a bus memory or bus memory controller, a peripheral bus, and a local bus that is able to interact with any other bus architecture. The system memory may include permanent memory (ROM) 1124 and random-access memory (RAM) 1125. The basic input/output system (BIOS) 1126 may store the basic procedures for transfer of information between elements of the computer system 1120, such as those at the time of loading the operating system with the use of the ROM 1124.

The computer system 1120 may also comprise a hard disk 1127 for reading and writing data, a magnetic disk drive 1128 for reading and writing on removable magnetic disks 1129, and an optical drive 1130 for reading and writing removable optical disks 1131, such as CD-ROM, DVD-ROM and other optical media. The hard disk 1127, the magnetic disk drive 1128, and the optical drive 1130 are connected to the system bus 1123 across the hard disk interface 1132, the magnetic disk interface 1133, and the optical drive interface 1134, respectively. The drives and the corresponding computer information media are power-independent modules for storage of computer instructions, data structures, program modules, and other data of the computer system 1120.

An example aspect comprises a system that uses a hard disk 1127 (which may store additional program applications 1187), a removable magnetic disk 1129 and a removable optical disk 1131 connected to the system bus 1123 via the controller 1155. It will be understood by those of ordinary skill in the art that any type of media 1156 that is able to store data in a form readable by a computer (solid state drives, flash memory cards, digital disks, random-access memory (RAM) and so on) may also be utilized.

The computer system 1120 has a file system 1136, in which the operating system 1135 may be stored, as well as program applications 1137, other program modules 1138, and program data 1139. A user of the computer system 1120 may enter commands and information using keyboard 1140, mouse 1142, or any other input device known to those of ordinary skill in the art, such as, but not limited to, a microphone, joystick, game controller, scanner, etc. Such input devices typically plug into the computer system 1120 through a serial port 1146, which in turn is connected to the system bus, but those of ordinary skill in the art will appreciate that input devices may be also be connected in other ways, such as, without limitation, via a parallel port, a game port, or a universal serial bus (USB). A monitor 1147 or other type of display device may also be connected to the system bus 1123 across an interface, such as a video adapter 1148. In addition to the monitor 1147, the personal computer may be equipped with other peripheral output devices (not shown), such as loudspeakers, a printer, etc.

Computer system 1120 may operate in a network environment, using a network connection to one or more remote computers 1149. The remote computer (or computers) 1149 may be local computer workstations or servers comprising most or all of the aforementioned elements in describing the nature of a computer system 1120. Other devices may also be present in the computer network, such as, but not limited to, routers, network stations, peer devices or other network nodes.

Network connections can form a local-area computer network (LAN) 1150 and a wide-area computer network (WAN). Such networks are used in corporate computer networks and internal company networks, and they generally have access to the Internet. In LAN or WAN networks, the computer system 1120 is connected to the local-area network 1150 across a network adapter or network interface 1151. When networks are used, the computer system 1120 may employ a modem 1154 or other modules well known to those of ordinary skill in the art that enable communications with a wide-area computer network such as the Internet. The modem 1154, which may be an internal or external device, may be connected to the system bus 1123 by a serial port 1146. It will be appreciated by those of ordinary skill in the art that said network connections are non-limiting examples of numerous well-understood ways of establishing a connection by one computer to another using communication modules.

In various aspects, the systems and methods described herein may be implemented in hardware, software, firmware, or any combination thereof. If implemented in software, the methods may be stored as one or more instructions or code on a non-transitory computer-readable medium. Computer-readable medium includes data storage. By way of example, and not limitation, such computer-readable medium can comprise RAM, ROM, EEPROM, CD-ROM, Flash memory or other types of electric, magnetic, or optical storage medium, or any other medium that can be used to carry or store desired program code in the form of instructions or data structures and that can be accessed by a processor of a general purpose computer.

In various aspects, the systems and methods described in the present disclosure can be addressed in terms of modules. The term “module” as used herein refers to a real-world device, component, or arrangement of components implemented using hardware, such as by an application specific integrated circuit (ASIC) or field-programmable gate array (FPGA), for example, or as a combination of hardware and software, such as by a microprocessor system and a set of instructions to implement the module’s functionality, which (while being executed) transform the microprocessor system into a special-purpose device. A module, element, or component may also be implemented as a combination of the two, with particular functions facilitated by hardware alone, and other functions facilitated by a combination of hardware and software. In particular implementations, at least a portion, and in some cases, all, of a module, element, or component may be executed on one or more processors of a general purpose computer. Accordingly, each module may be realized in a variety of suitable configurations, and should not be limited to any particular implementation or example herein. An element, or any portion of an element, or any combination of elements may be implemented as a “processing system” that includes one or more processors. When multiple processors are implemented, the multiple processors may perform the functions individually or in combination. One or more processors in a processing system may execute stored instructions, which may be referred to as software, firmware, middleware, microcode, hardware description language, or otherwise,e.g., instructions, instruction sets, code, code segments, program code, programs, subprograms, software components, applications, software applications, software packages, routines, subroutines, objects, executables, threads of execution, procedures, functions, or any combination thereof.

In one configuration, the scope position component 1175 and/or the computer system 1120, and in particular, the file system 1136 and/or the processor (e.g., 1121), is configured to perform the aspects of any of FIGS. 1-5, 9A, 9B, and 10. In some aspects, the scope position component 1175 may correspond to one or more of the scope position component 538 and/or the visual indication component 540 of the control unit 575 illustrated in FIG. 5.

FIG. 12 illustrates example aspects of model implementations and training, in accordance with aspects presented herein. FIG. 12 is described with reference to FIG. 5, including the model 590, the control unit 575, the input component 520, and the output component 522. FIG. 14 illustrates a related example showing the input of historical data for training the model 590 and the input of inference data for a particular patient, prior to or during a procedure, in order to obtain an inference output from the trained model 590.

With respect to training / refining the model 590, modeling data 1217 may be utilized and may include historical information, a priori information, training information, and/or the like, e.g., data and information gathered / collected prior to a current endoscopic procedure. The modeling data 1217 may be pre-programmed and/or received via the input component 520 as training inputs, according to aspects. The modeling data 1217 may include historical data from multiple endoscopy procedures. For example for each prior endoscopy procedure, the modeling data 1217 may include a set of information that includes one or more of endoscope positioning information 1202, endoscope positioning information 1204, endoscopic procedure information 1206 for a set of completed endoscopy procedures, anatomical information 1208 associated with one or more of the set of completed endoscopy procedures, patient information 1210 associated with one or more of the set of completed endoscopy procedures, endoscopy device information 1212 associated with one or more of the set of completed endoscopy procedures, endoscope information 1214 associated with one or more of the set of completed endoscopy procedures, medical personnel information 1216 associated with one or more of the set of completed endoscopy procedures, and/or the like.

For example, various types of data or information can be generated by, captured, and/or used by robotic endoscope devices. The information generated by and/or captured by the endoscope may be provided as input to the model, e.g., such as in FIG. 12, to obtain inference output to assist in the procedure, including to assist in compression device adjustment (whether automatic or assisting a user in making a manual or other type of user assisted adjustment). As an example, the data or information generated by, captured, and/or used by robotic endoscope devices may include visual imagery and/or video captured by the camera at the endoscope. As an example, the image information may be via while-light imaging, complimentary metal-oxide semiconductor sensors, as well as narrow band imaging, blue light imaging, and/or autofluorescence features. As an example, the data or information generated by, captured, and/or used by robotic endoscope devices may include position or orientation information, such as provide by one or more position sensors, orientation sensors, accelerometers, gyroscopes, and/or magnetometers. The sensors may be located at the endoscope, for example, and/or may make a measurement based on the endoscope location/orientation. As an example, the data or information generated by, captured, and/or used by robotic endoscope devices may include electromagnetic sensors. As an example, the data or information generated by, captured, and/or used by robotic endoscope devices may include force and pressure sensors, such as strain gauges, balloon based pressure sensors, and/or fiber-optic force sensors that measure axial force, lateral force, and/or torque. As an example, the data or information generated by, captured, and/or used by robotic endoscope devices may include shape and/or bending sensors, such as fiber Bragg grating sensors that measure strain along the shaft of the endoscope during the endoscopy procedure. As an example, the data or information generated by, captured, and/or used by robotic endoscope devices may include optical flow sensors, such as sensors that combine video imagery with algorithms to determine movement direction and distance, relative direction, and/or lumen centering associated with the endoscope. As an example, the data or information generated by, captured, and/or used by robotic endoscope devices may include tissue characterization sensors such as optical coherence tomography, ultrasound micro transducers, and/or electrical impedance sensors. As an example, the data or information generated by, captured, and/or used by robotic endoscope devices may include sensors such as motor current, temperature, and/or cable tension sensors. The data or information generated by, captured, and/or used by robotic endoscope devices may include any combination of such data capturing devices and/or sensors. In some aspects, the obtained data may be provided as the input (e.g., shown at 520) to the model (e.g., 590). Based on the input information, the model may provide an inference regarding a compression adjustment that would assist the endoscopy procedure, e.g., to improve movement of the endoscope and/or imaging obtained via the endoscope. Similarly, any of the described examples of data may be used as training data to train and/or refine the model to provide improved endoscopic results through targeted abdominal compression adjustment.

In some aspects, the endoscope may have wired controls, e.g., the mechanical controls that enable a physician or system to control movement of the endoscope. The data may be received from the sensors at the endoscope via the wired controls. The sensor data may then be provided to the model 590 via a communication interface.

Any subset, any combination, or all of these types of data could be used to adjust the external compression device to optimally assist scope movement and/or to provide stabilization to support better visualization and/or the delivery of various therapeutic endoscopic tasks and procedures.

In some aspects, the output may instruct, control, or drive a fully-autonomous robotic system. In some aspects, the output may provide inference information for robotic-assisted endoscopic procedures. In such examples, the output may include instruction to adjust an external pressure device, e.g., where the adjustment is predicted to avoid a second complication during the endoscopic procedure. As an example, the output may include a flag identifying an observed loop or predicting a potential loop at the patient during the endoscopic procedure and/or a recommendation of an action that is predicted to avoid a first complication during the endoscopic procedure. In such robotic assisted procedures, a user may provide additional or alternative input or control that results in adjustments of the compression device. The inputs may be received from the user via handheld joysticks or other controller inputs which adjust an external compression device and/or scope movement. In some aspects, the user received control may simultaneously direct outputs of scope and external compression device. The user inputs may be received via foot pedals, touch screen interfaces, haptic feedback devices, voice commands, and/or other input reception interfaces. The system may include an override function or bypass design where physician inputs go directly to the external compression device without interacting with scope, thus giving physician exclusive control over external compression mechanisms. In such a design, haptic, visual, or auditory feedback mechanisms to indicate the magnitude and location of pressure being delivered to the patient by the compression device can be helpful. In such examples, the output of the model may provide suggestions for amounts and/or locations of compressions via visual output and/or audio output.

In aspects, the endoscopic procedure information may include data associated with at least one of: an anticipated path of the endoscope, a first set of prior positional references associated with looping in a set of prior endoscopic procedures, a second set of prior positional references associated with prior applications of pressure during the set of prior endoscopic procedures, and/or the like.

In aspects, for one or more endoscopy procedures in the set of completed endoscopy procedures on which the model 590 is based, the anatomical information may include data associated with at least one of: positioning of a set of one or more anatomical features of a corresponding patient, size of the set of one or more anatomical features of the corresponding patient, shape of the set of one or more anatomical features of the corresponding patient, location of the set of one or more anatomical features of the corresponding patient, a health assessment associated with the corresponding patient, and/or the like.

In aspects, for one or more endoscopy procedures in the set of completed endoscopy procedures on which the model is based, the patient information may include data associated with at least one of: a first orientation of a corresponding patient with respect to the endoscope, a second orientation of the corresponding patient with respect to a display for visual output associated with the endoscopic procedure, a set of patient dimensional measurements, a medical history of the corresponding patient, and/or the like.

In aspects, for one or more endoscopy procedures in the set of completed endoscopy procedures on which the model is based, the endoscopy device information may include data associated with at least one of: a manufacturer of the endoscopy device used in a corresponding endoscopy procedure, a model type of the endoscopy device used in the corresponding endoscopy procedure, a set of adjustable pressure capabilities of the endoscopy device used in the corresponding endoscopy procedure, a set of visual output capabilities of the endoscopy device used in the corresponding endoscopy procedure, a set of sensor capabilities of the endoscopy device used in the corresponding endoscopy procedure, a set of communication capabilities of the endoscopy device used in the corresponding endoscopy procedure, and/or the like.

In aspects, for one or more endoscopy procedures in the set of completed endoscopy procedures on which the model is based, the endoscope information may include data associated with at least one of: a manufacturer of the endoscope used in a corresponding endoscopy procedure, a model type of the endoscope used in the corresponding endoscopy procedure, a set of movement capabilities of the endoscope used in the corresponding endoscopy procedure, signal emitter information of the endoscope used in the corresponding endoscopy procedure, a set of feedback information types of which the endoscope is configured to provide, and/or the like.

In aspects, for one or more endoscopy procedures in the set of completed endoscopy procedures on which the model is based, the medical personnel information may include data associated with a set of endoscopic procedure preferences of medical personnel, comprising at least one of: an amount of pressure applied for looping prevention in a corresponding endoscopy procedure, a set of patient orientations for the corresponding endoscopy procedure, a display of visual output for the corresponding endoscopy procedure, a manufacturer of the endoscope used in the corresponding endoscopy procedure, a model type of the endoscope used in the corresponding endoscopy procedure, and/or the like.

FIG. 14 illustrates an example of the input of data for model training and then for model inference. For example, FIG. 14 illustrates that historical data may be input to the model providing various types of endoscopy procedure information, and outcome information. Then, for a particular endoscopy procedure, a set of patient specific information may be input to the model (e.g., as inference input data) to obtain an inference or model output from the model that was previously trained on the historical endoscopy data. For a particular patient, the input data may include one or more of endoscope positioning information 1402, endoscope positioning information 1404, endoscopic procedure information 1406 for the current endoscopy procedures anatomical information 1408 associated with the current patient, patient information 1410 for the current patient, endoscopy device information 1412 of the device that will be used in the current procedure, endoscope information 1414 of the endoscope that will be used in the current procedure, medical personnel information 1416 identifying one or more medical professionals that will be involved with the current endoscopy procedure, and/or the like.

As an example, the current endoscopic procedure information for the current patient (e.g., the inference data input) may include data associated with at least one of: an anticipated path of the endoscope, position information for the endoscope, current application of pressure, and/or the like. In some aspects, the endoscope position information may include magnetic imaging information, such as described in connection with FIG. 6.

As an example, the anatomical information for the current patient (e.g., the inference data input) may include data associated with at least one of: positioning of a set of one or more anatomical features of the patient, size of the set of one or more anatomical features of the patient, shape of the set of one or more anatomical features of the patient, location of the set of one or more anatomical features of the patient, a health assessment associated with the patient, and/or the like.

As an example, the patient information for the current patient (e.g., the inference data input) may include data associated with at least one of: a first orientation of a corresponding patient with respect to the endoscope, a second orientation of the corresponding patient with respect to a display for visual output associated with the endoscopic procedure, a set of patient dimensional measurements, a medical history of the corresponding patient, and/or the like.

As an example, the endoscopy device information for the current patient (e.g., the inference data input) may include data associated with at least one of: a manufacturer of the endoscopy device for the endoscopy procedure, a model type of the endoscopy device for the endoscopy procedure, a set of adjustable pressure capabilities of the endoscopy device for the endoscopy procedure, a set of visual output capabilities of the endoscopy device for the endoscopy procedure, a set of sensor capabilities of the endoscopy device for the endoscopy procedure, a set of communication capabilities of the endoscopy device for the endoscopy procedure, and/or the like.

As an example, the endoscope information for the current patient (e.g., the inference data input) may include data associated with at least one of: a manufacturer of the endoscope for the endoscopy procedure, a model type of the endoscope for the endoscopy procedure, a set of movement capabilities of the endoscope for the endoscopy procedure, signal emitter information of the endoscope for the endoscopy procedure, a set of feedback information types of which the endoscope is configured to provide, and/or the like.

The feedback illustrated in FIG. 14 may include feedback that is used to train the model, as well as ongoing feedback relating to the current status of a current procedure, e.g., which may be used to provide a new or refined inference (e.g., identification of a potential complication, recommendation to avoid the potential complication, and/or automatic adjustment of pressure or movement of the endoscope).

In aspects, outcome / feedback information 1218 may also be utilized as training data, via the input component 520, to train / refine (e.g., via a training component 1291) the model 590. The outcome / feedback information 1218 may include an outcome / feedback of a medical result 1218a associated with an output 1218b (e.g., 532 of FIG. 5), e.g., one or more of the flag identifying the potential loop, the recommendation of the action, or the instruction to adjust the external pressure device. In some aspects, the outcome / feedback information 1218 may also include endoscope feedback information, e.g., via a wired connection or a wireless connection with the endoscope. In some aspects, the endoscope feedback information may include additional information associated with the endoscope, comprising at least one of orientation, flexion, or applied force. In some aspects, the outcome / feedback information 1218 may also include data / information from other types of medical equipment, camera images / frames, voice inputs, and/or the like.

The model 590 may be refined using feedback from multiple endoscopy procedures performed by multiple medical professionals. For example, the model 590 may be a central model that receives feedback from multiple users based on use of the model during various endoscopy procedures. This may enable the model to provide inferences based on modeling from a broad group of patients and medical professionals. In some aspects, the model 590 may be a local model that is refined for a particular subset of one or more medical professionals. For example, the subset may correspond to a medical group of multiple medical professionals. In some aspects, the subset may correspond to a single medical professional, and the model may be refined based on feedback for that particular medical professional in order to improve the inferences output may the model when used in connection with endoscopy procedures performed by that particular medical professional. The refinement based on feedback for a particular medical professional, for example, may enable the model 590 to provide more accurate inferences and predictions of potential looping, recommended interventions that have been successful for that particular medical professional, and/or to automatically adjust compression in a way that is targeted to assisting the particular medical professional.

The model 590 may undergo training / refining (e.g., via a training component 1291) based on the modeling data 1217 (e.g., historical information from before an endoscopic procedure) and/or the outcome / feedback information 1218 (e.g., current information during an endoscopic procedure). That is, the training component 1291 may train / refine the model 590 before and/or during an endoscopic procedure.

In aspects, the input component 520 may be configured to receive, as model inputs during execution of the model 590, one or more of the modeling data 1217, and may be configured to receive, as model inputs, one or more of the outcome / feedback information 1218. Accordingly, the model 590 may be configured to output an inference 1299, based on the training / refining (e.g., via a training component 1291) and the model inputs, when executed by the control unit 575 during an endoscopic procedure. In aspects, the control unit 575 may be configured to activate / deactivate, for self-adjustment, an application of adjustable pressure (as described herein, e.g., for FIG. 5) based on the inference 1299 of the model 590, during an endoscopic procedure.

As one example, the control unit 575 may be configured to activate (at 1260), for self-adjustment and in accordance with the instruction from the output component, an application of the adjustable pressure through the compression for the endoscopic procedure based on the information, and to deactivate (at 1262), for the self-adjustment and in accordance with the instruction from the output component, the application of the adjustable pressure through lessening of the compression for the endoscopic procedure based on the information. As one example, the control unit 575 may be configured to determine (at 1264) at least one of a presence of looping or a looping prediction during the endoscopic procedure based on the information and in accordance with the model, and to activate (at 1266), for the self-adjustment and in accordance with the model, the application of the adjustable pressure through the compression for the endoscopic procedure based on the information and at a position relative to the at least part of the abdomen of the patient corresponding to the presence of looping or the looping prediction. As one example, the control unit 575 may be configured to activate (at 1268), for the self-adjustment and in accordance with the model, the application of the adjustable pressure at a specific level of pressure through the compression for the endoscopic procedure based on the information. As one example, the control unit 575 may be configured to activate (at 1270), for the self-adjustment and in accordance with the model, the application of the adjustable pressure through the compression for the endoscopic procedure based on the information and on endoscope feedback information.

In aspects, the control unit 575 may be configured to receive, via the input component 520, an override input indication 1252 indicative of a termination of the adjustable pressure. Accordingly, the control unit 575 may be configured to deactivate the application of the adjustable pressure (e.g., via a deactivation override output 1254) based on the override input indication 1252 being received.

In some aspects, the input 520 and/or the feedback 1218 may include pressure (or other metrics) measured via sensors placed at the patient’s abdomen. For example, one or more pressure sensors may be placed between the patient and the device that applies/adjusts compression at the patient’s abdomen. In some aspects, the sensor(s) may be comprised within the compression device. The compression may be adjusted via one or more of various mechanisms. For example, the device may include an array of inflatable bladders that can be adjusted, e.g., the size of which can be increased through filling the bladder with air or fluid to increase pressure, or reduced through removal of air or fluid to decrease pressure. As another example, the compression may be adjusted via one or more straps that are pulled (e.g., retracted) to increase compression/pressure over a particular area of the patient’s abdomen or released (e.g., extended) to reduce the compression/pressure. As another example, the compression may be applied using a material within the compression device that changes form based on application of a signal. For example, the compression device may include a foam material (or other material) having a variable rigidity that can be adjusted based on an application of current to the material. The array of bladders allows targeted pressure/compression to be applied to particular areas of the patient’s abdomen. For example, the model 590 may predict a potential for looping at a location (e.g., a location near the endoscope position) and may apply added pressure at that area or in a predicted path of the endoscope to assist in preventing the potential looping. In another example, the pressure may be applied by robotic arms that are directed based on output from the model. Similarly, the model may be used to indicate, recommend, and in certain instances direct adjustments to the height and/or orientation of bed or stretcher on which the patient is lying in order to adjust the patient’s positioning, for the purposes of augmenting compression and controlling looping.

In some aspects, the endoscope may be self-propelled in connection with inferences from the model. The feedback from the endoscope may be used, e.g., in connection with additional sensor information to identify the potential for looping or other complications and to coordinate the movement of the endoscope with the targeted application of compression to reduce or avoid the potential looping identified by the model. This enables the control unit for the movement of the endoscope to coordinate control of the compression device (e.g., as a shared control unit or by communicating with a control unit of the compression device), in some aspects. In other aspects, the information from the endoscope may be used to recommend interventions (such as application of targeted pressure, movement of the patient, etc.) to a medical professional, who may then determine whether to implement the recommended intervention.

Over time, the model 590 may predict, or identify sequences of endoscopy actions (which may include a sequence of compression application including various magnitudes of compression/pressure application) to optimize endoscopy procedure outcomes. The identified sequence, or pattern, may be provided to a medical professional in advance of the endoscopy procedure as a tool to assist with preparation for the endoscopy procedure. The identified sequence may be used to control/instruct one or more automated components (such as a self-propelled endoscope and/or self-adjusting compression device). In some aspects, the identified sequence may be specific to a particular medical professional based on outcome feedback (or other procedure feedback) from a history of prior endoscopy procedures performed by that particular medical professional. For example, the model may be trained based on a broad set of training data from endoscopy procedures for multiple medical professionals, and then may be refined to provide assistance to a particular medical professional based on a combination of the training data and feedback for the particular medical professional. In other aspects, the model may continue to be refined based on feedback information from multiple medical professionals, e.g., which may enable optimized assistance based on the more favorable outcome feedback across multiple medical professionals.

The assistance of the model 590, whether through recommendations, identification of potential complications, and/or automated assistance through a compression device or self-propelled endoscope, can help to improve the efficiency of the endoscopy procedures, improving outcomes and reducing complications to patients and injuries to medical professionals.

As an example, the automated application of targeted pressure enables a more consistent application of compression based on sensor driven identification of potential complications, which can be applied/adjusted more efficiently than by a person deciding to manually make an adjustment. Some aspects may enable a medical professional to assist with an endoscopy procedure via a remote connection to one or more automated components. This may enable increased access to medical assistance at locations that are distant from medical professionals that specialize in a needed endoscopy procedure. As illustrated at 1151 in FIG. 11, or 542 in FIG. 5, the system may include a communication interface that allows communication with one or more remote computers. For example, such a communication interface may enable a specialist at a remote location to work with local medical professionals in connection with an endoscopy procedure for a local patient.

In some aspects, the model 590 may use machine-learning algorithms, deep-learning algorithms, neural networks, reinforcement learning, regression, boosting, or advanced signal processing methods for predicting looping, predicting an intervention to reduce or avoid potential looping or other complications, and/or automatically adjusting compression via a compression device for a particular endoscopy procedure for a particular endoscopy patient.

Various aspects of artificial intelligence or machine learning may be implemented in the model 590. As an example, reinforcement learning is a type of machine learning that involves the concept of taking actions in an environment in order to maximize a reward. Reinforcement learning is a machine learning paradigm. Other paradigms include supervised learning and unsupervised learning. Basic reinforcement may be modeled as a Markov decision process (MDP) with a set of environment states and agent states, as well as a set of actions of the agent. A determination may be made about a likelihood of a state transition based on an action and a reward after the transition. The action selection by an agent may be modeled as a policy. The reinforcement learning may enable the agent to learn an optimal, or nearly-optimal, policy that maximizes a reward. Supervised learning may include learning a function that maps an input to an output based on example input-output pairs, which may be inferred from a set of training data, which may be referred to as training examples. The supervised learning algorithm analyzes the training data and provides an algorithm to map to new examples.

Regression analysis may include statistical analysis to estimate the relationships between a dependent variable (e.g., an outcome variable) and one or more independent variables. Linear regression is an example of a regression analysis. Non-linear regression models may also be used. Regression analysis may include estimating, or determining, relationships of cause between variables in a dataset.

Boosting includes one or more algorithms for reducing variance or bias in supervised learning. Boosting may include iterative learning based on weak classifiers (e.g., that are somewhat correlated with a true classification) with respect to a distribution that is added to a strong classifier (e.g., that is more closely correlated with the true classification) in order to convert weak classifiers to stronger classifiers. The data weights may be readjusted through the process, e.g., related to accuracy.

Among others, examples of machine learning models or neural networks that may be included in the AI/ML model include, for example, artificial neural networks (ANN); decision tree learning; convolutional neural networks (CNNs); deep learning architectures in which an output of a first layer of neurons becomes an input to a second layer of neurons, and so forth; support vector machines (SVM), e.g., including a separating hyperplane (e.g., decision boundary) that categorizes data; regression analysis; Bayesian networks; genetic algorithms; deep convolutional networks (DCNs) configured with additional pooling and normalization layers; and deep belief networks (DBNs).

In some aspects, an example machine learning model, such as an artificial neural network (ANN), that includes an interconnected group of artificial neurons (e.g., neuron models) as nodes. Neuron model connections may be modeled as weights, in some aspects. A machine learning model may be adapted, e.g., based on external or internal information processed by the machine learning model. In some aspects, a machine learning model may include a non-linear statistical data model and/or a decision making model. Machine learning may model complex relationships between input data and output information.

A machine learning model may include multiple layers and/or operations that may be formed by concatenation of one or more of the referenced operations. Examples of operations that may be involved include extraction of various features of data, convolution operations, fully connected operations that may be activated or deactivated, compression, decompression, quantization, flattening, etc. The term layer may indicate an operation on input data. Weights, biases, coefficients, and operations may be adjusted in order to achieve an output closer to the target output. Weights and biases are examples of parameters of a trained machine learning model. Different layers of a machine learning model may be trained separately.

A variety of connectivity patterns, e.g., including any of feed-forward networks, hierarchical layers, recurrent architectures, feedback connections, etc., may be included in a machine learning model. Layer connections may be fully connected or locally connected. For a fully connected network, a first layer neuron may communicate an output to each neuron in a second layer. Each neuron in the second layer may receive input from each neuron in the first layer. For a locally connected network, a first layer neuron may be connected to a subset of neurons in the second layer, rather than to each neuron of the second layer. A convolutional network may be locally connected and may be configured with shared connection strengths associated with the inputs for each neuron in the second layer. In a locally connected layer of a network, each neuron in a layer may have the same, or a similar, connectivity pattern, yet having different connection strengths.

A machine learning model, artificial intelligence component, or neural network may be trained, such as training based on supervised learning. During training, the machine learning model may be presented with an input that the model uses to compute to produce an output. The actual output may be compared to a target output, and the difference may be used to adjust parameters (e.g., weights, biases, coefficients, etc.) of the machine learning model in order to provide an output closer to the target output. Before training, the output may not be correct or may be less accurate. A difference between the output and the target output, may be used to adjust weights of a machine learning model to align the output is more closely with the target.

A learning algorithm may calculate a gradient vector for adjustment of the weights. The gradient may indicate an amount by which the difference between the output and the target output would increase or decrease if the weight were adjusted. The weights, biases, or coefficients of the model may be adjusted until an achievable error rate stops decreasing or until the error rate has reached a target level.

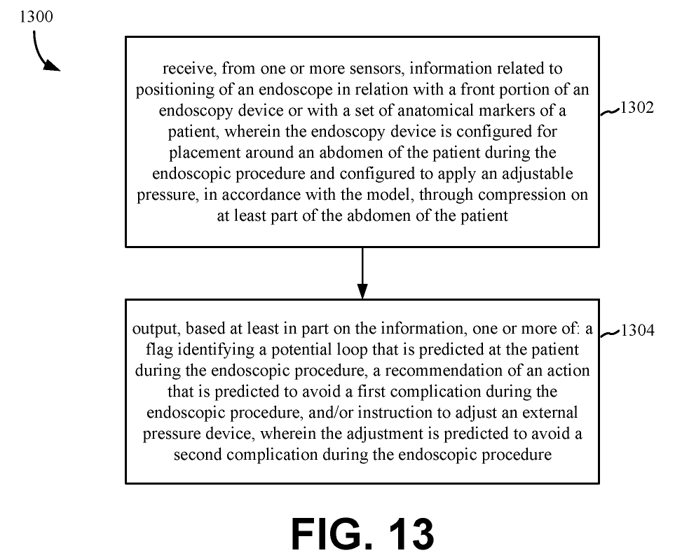

FIG. 13 illustrates an example flowchart 1300 showing a method utilizing a model to support an endoscopic procedure, with an endoscopy device configured for placement at an abdomen of a patient during the endoscopic procedure.

As shown at 1302, the method includes receiving, via an input component and from one or more sensors, information related to positioning of an endoscope in relation with a front portion of an endoscopy device or with a set of anatomical markers of a patient. In aspects, the endoscopy device may be configured for placement around an abdomen of the patient during the endoscopic procedure and configured to apply an adjustable pressure, in accordance with the model, through compression on at least part of the abdomen of the patient. The endoscopy device may include any of the aspects described in connection with FIGS. 1-12. In some aspects, the endoscopy device may be a compression device.

At 1304, the method includes outputting, via an output component and based at least in part on the information, one or more of: a flag identifying a potential loop that is predicted at the patient during the endoscopic procedure, a recommendation of an action that is predicted to avoid a first complication during the endoscopic procedure, and/or instruction to adjust an external pressure device, wherein the adjustment is predicted to avoid a second complication during the endoscopic procedure.

In some aspects, visual output includes: activating one or more LEDs of an LED array provided on the endoscopy device to correspond with the relative position and a shape of the endoscope. In some aspects, changing the visual output includes outputting to a display a visual indication of the position of the endoscope relative to the set of anatomical landmarks displayed at the display, and wherein the display is separate from the endoscopy device, and wherein the endoscopy device includes a reference component that provides a reference for the anatomical landmarks relative to the positioning of the endoscope.

In some aspects, the method may further include detecting, via the one or more sensors, one or more magnets of the endoscope during the endoscopic procedure; and identifying, based on information from the one or more sensors, the relative position of the endoscope relative to the front portion of the endoscopy device or relative to the set of anatomical landmarks of the patient.

In some aspects, the one or more sensors are configured to detect the one or more magnets of the endoscope as the endoscope traverses a colon of the patient. In some aspects, the one or more sensors are configured to provide the information related to the positioning of the endoscope in relation with the set of anatomical landmarks of the patient. In some aspects, the method includes aligning the one or more sensors are with the set of anatomical landmarks of the patient, and wherein the positioning of the endoscope is detected in relation to the one or more sensors.

In some aspects, the method includes providing a visual pressure indication in relation with the set of anatomical landmarks of the patient, wherein the pressure indication indicates a location for external pressure on the abdomen of the patient.

In some aspects, the visual indication component may be a LED array provided on the endoscopy device, wherein one or more LEDs of the LED array are configured to activate to correspond with the relative position and a shape of the endoscope. In some aspects, the method includes activating in a visual indication, associated with the relative position of the endoscope, to correspond with at least one of: a presence or a potential presence of looping, or a location to apply a pressure through compression on at least part of the abdomen of the patient.

In some aspects, the endoscopy device is a compression device, and the method further includes applying the pressure through the compression through application of the device on at least part of the abdomen of the patient that corresponds to the location and the visual indication. In some aspects, the method includes activating the one or more LEDs of the LED array in a different visual indication, associated with the positioning of the endoscope, to correspond with an absence of looping.

In some aspects, the method includes outputting to a display a visual indication of the position of the endoscope relative to an outline of the patient displayed at the display, wherein the display is separate from the endoscopy device.

Aspects herein may be performed in accordance with or based on a model, as described above.

Example aspects have now been described in accordance with the above advantages. It will be appreciated that these examples are merely illustrative of aspects, and many variations and modifications will be apparent to those skilled in the art.

It is understood that the specific order or hierarchy of steps in the processes disclosed is an illustration of example approaches. Based upon design preferences, it is understood that the specific order or hierarchy of steps in the processes may be rearranged. Further, some steps may be combined or omitted. The accompanying method claims present elements of the various steps in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects.” Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

The following aspects are illustrative only and may be combined with other aspects or teachings described herein, without limitation.

Aspect 1 is an apparatus utilizing a model to support an endoscopic procedure, comprising: an input component configured to: receive, from one or more sensors, information related to positioning of an endoscope in relation with a front portion of an endoscopy device or with a set of anatomical markers of a patient, wherein the endoscopy device is configured for placement around an abdomen of the patient during the endoscopic procedure and configured to apply an adjustable pressure, in accordance with the model, through compression on at least part of the abdomen of the patient; and an output component configured to output, based at least in part on the information, one or more of: a flag identifying an observed loop or predicting a potential loop at the patient during the endoscopic procedure, a recommendation of an action that is predicted to avoid a first complication during the endoscopic procedure, or instruction to adjust an external pressure device, wherein the adjustment is predicted to avoid a second complication during the endoscopic procedure.

In aspect 2, the apparatus of aspect 1 further includes a control unit configured to: activate, for self-adjustment and in accordance with the instruction from the output component, an application of the adjustable pressure through the compression for the endoscopic procedure based on the information.

In aspect 3, the apparatus of aspect 2 further includes that the control unit is configured to: deactivate, for the self-adjustment and in accordance with the instruction from the output component, the application of the adjustable pressure through lessening of the compression for the endoscopic procedure based on the information.

In aspect 4, the apparatus of aspect 2 or aspect 3 further includes that the control unit is further configured to: determine at least one of a presence of looping or a looping prediction during the endoscopic procedure based on the information and in accordance with the model; wherein the control unit is configured to activate, for the self-adjustment and in accordance with the model, the application of the adjustable pressure through the compression for the endoscopic procedure based on the information and at a position relative to the at least part of the abdomen of the patient corresponding to the presence of looping or the looping prediction.

In aspect 5, the apparatus of any of aspects 2-4 further includes that the control unit is configured to: activate, for the self-adjustment and in accordance with the model, the application of the adjustable pressure at a specific level of pressure through the compression for the endoscopic procedure based on the information, or wherein the control unit is configured to activate, for the self-adjustment and in accordance with the model, the application of the adjustable pressure through the compression for the endoscopic procedure based on the information and on endoscope feedback information.

In aspect 6, the apparatus of aspect 5 further includes that the input component is further configured to: receive the endoscope feedback information via a wired connection or a wireless connection with the endoscope; and/or wherein the endoscope feedback information includes additional information associated with the endoscope, comprising at least one of orientation, flexion, or applied force.

In aspect 7, the apparatus of any of aspects 2-6 further includes that the input component is further configured to: receive an override input indication indicative of a termination of the adjustable pressure; wherein the control unit is further configured to: deactivate the application of the adjustable pressure based on the override input indication being received.

In aspect 8, the apparatus of any of aspects 1-7 further includes that the model is based on modeling data comprising at least one of: endoscopic procedure information for a set of completed endoscopy procedures, anatomical information associated with one or more of the set of completed endoscopy procedures, patient information associated with one or more of the set of completed endoscopy procedures, endoscopy device information associated with one or more of the set of completed endoscopy procedures, endoscope information associated with one or more of the set of completed endoscopy procedures, or medical personnel information associated with one or more of the set of completed endoscopy procedures.

In aspect 9, the apparatus of aspect 8 further includes that the model is an artificial intelligence (AI) / machine learning (ML) (AI/ML) model, wherein the modeling data is training data on which the AI/ML model is based.

In aspect 10, the apparatus of aspect 8 or aspect 9 further includes that the endoscopic procedure information includes data associated with at least one of: an anticipated path of the endoscope, a first set of prior positional references associated with looping in a set of prior endoscopic procedures, or a second set of prior positional references associated with prior applications of pressure during the set of prior endoscopic procedures.

In aspect 11, the apparatus of any of aspects 8-10 further includes that, for one or more endoscopy procedures in the set of completed endoscopy procedures on which the model is based, the anatomical information includes data associated with at least one of: positioning of a set of one or more anatomical features of a corresponding patient, size of the set of one or more anatomical features of the corresponding patient, shape of the set of one or more anatomical features of the corresponding patient, location of the set of one or more anatomical features of the corresponding patient, or a health assessment associated with the corresponding patient.

In aspect 12, the apparatus of any of aspects 8-11 further includes that, for one or more endoscopy procedures in the set of completed endoscopy procedures on which the model is based, the patient information includes data associated with at least one of: a first orientation of a corresponding patient with respect to the endoscope, a second orientation of the corresponding patient with respect to a display for visual output associated with the endoscopic procedure, a set of patient dimensional measurements, or a medical history of the corresponding patient.

In aspect 13, the apparatus of any of aspects 8-12 further includes that, for one or more endoscopy procedures in the set of completed endoscopy procedures on which the model is based, the endoscopy device information includes data associated with at least one of: a manufacturer of the endoscopy device used in a corresponding endoscopy procedure, a model type of the endoscopy device used in the corresponding endoscopy procedure, a set of adjustable pressure capabilities of the endoscopy device used in the corresponding endoscopy procedure, a set of visual output capabilities of the endoscopy device used in the corresponding endoscopy procedure, a set of sensor capabilities of the endoscopy device used in the corresponding endoscopy procedure, or a set of communication capabilities of the endoscopy device used in the corresponding endoscopy procedure.

In aspect 14, the apparatus of any of aspects 8-13 further includes that, for one or more endoscopy procedures in the set of completed endoscopy procedures on which the model is based, the endoscope information includes data associated with at least one of: a manufacturer of the endoscope used in a corresponding endoscopy procedure, a model type of the endoscope used in the corresponding endoscopy procedure, a set of movement capabilities of the endoscope used in the corresponding endoscopy procedure, signal emitter information of the endoscope used in the corresponding endoscopy procedure, or a set of feedback information types of which the endoscope is configured to provide.

In aspect 15, the apparatus of any of aspects 8-14 further includes that, for one or more endoscopy procedures in the set of completed endoscopy procedures on which the model is based, the medical personnel information includes data associated with a set of endoscopic procedure preferences of medical personnel, comprising at least one of: an amount of pressure applied for looping prevention in a corresponding endoscopy procedure, a set of patient orientations for the corresponding endoscopy procedure, a display of visual output for the corresponding endoscopy procedure, a manufacturer of the endoscope used in the corresponding endoscopy procedure, or a model type of the endoscope used in the corresponding endoscopy procedure.

In aspect 16, the apparatus of any of aspects 1-15 further includes a training component, wherein the training component is configured to: receive at least one of outcome information or feedback of a medical result associated with the output of one or more of the flag identifying the potential loop, the recommendation of the action, or the instruction to adjust the external pressure device; and train or refine the model based on at least one of the outcome information or the feedback.

In aspect 17, the apparatus of any of aspects 1-16 further includes that the input component is further configured to receive, as model input at least one of: endoscopic procedure information for the endoscopic procedure for the patient, anatomical information associated with the endoscopic procedure for the patient, patient information for the patient, endoscopy device information about the endoscopy device associated with the endoscopic procedure, endoscope information for the endoscope associated with the endoscopic procedure, or medical personnel information for one or more persons that will participate in the endoscopic procedure for the patient; and wherein model output from the model is an inference based on the model input.

In aspect 18, the apparatus of any of aspects 1-17 further includes a visual indication component configured to: change visual output, for at least one of a display or light emitting diode (LED) array associated with the endoscopy device, relative to a position of the endoscope and in accordance with the model.

In aspect 19, the apparatus of aspect 18 further includes that the visual output comprises at least one of: a first indication of a presence of looping associated with the endoscopic procedure; a second indication of a looping prediction associated with the endoscopic procedure; a third indication of an absence of looping associated with the endoscopic procedure; or a fourth indication of a pressure position relative to the at least part of the abdomen of the patient corresponding to the presence of looping or the looping prediction for an application of the adjustable pressure.

Aspect 20 is a method utilizing a model to support an endoscopic procedure, comprising: receiving, via an input component and from one or more sensors, information related to positioning of an endoscope in relation with a front portion of an endoscopy device or with a set of anatomical markers of a patient, wherein the endoscopy device is configured for placement around an abdomen of the patient during the endoscopic procedure and configured to apply an adjustable pressure, in accordance with the model, through compression on at least part of the abdomen of the patient; and outputting, via an output component and based at least in part on the information, one or more of: a flag identifying an observed loop or a potential loop that is predicted at the patient during the endoscopic procedure, a recommendation of an action that is predicted to avoid a first complication during the endoscopic procedure, or instruction to adjust an external pressure device, wherein the adjustment is predicted to avoid a second complication during the endoscopic procedure.

In aspect 21, the method of aspect 20 further includes activating, for self-adjustment and in accordance with the instruction from the output component, an application of the adjustable pressure through the compression for the endoscopic procedure based on the information.

In aspect 22, the method of aspect 21 further includes deactivating the self-adjustment and in accordance with the instruction from the output component, the application of the adjustable pressure through lessening of the compression for the endoscopic procedure based on the information.

In aspect 23, the method of aspect 21 or aspect 22 further includes determining at least one of a presence of looping or a looping prediction during the endoscopic procedure based on the information and in accordance with the model; wherein the control unit is configured to activate, for the self-adjustment and in accordance with the model, the application of the adjustable pressure through the compression for the endoscopic procedure based on the information and at a position relative to the at least part of the abdomen of the patient corresponding to the presence of looping or the looping prediction.

In aspect 24, the method of any of aspects 21-23 further includes activating, for the self-adjustment and in accordance with the model, the application of the adjustable pressure at a specific level of pressure through the compression for the endoscopic procedure based on the information, or wherein the control unit is configured to activate, for the self-adjustment and in accordance with the model, the application of the adjustable pressure through the compression for the endoscopic procedure based on the information and on endoscope feedback information.

In aspect 25, the method of aspect 24 further includes receiving the endoscope feedback information via a wired connection or a wireless connection with the endoscope; and/or wherein the endoscope feedback information includes additional information associated with the endoscope, comprising at least one of orientation, flexion, or applied force.

In aspect 26, the method of any of aspects 21-25 further includes receiving an override input indication indicative of a termination of the adjustable pressure; and deactivating the application of the adjustable pressure based on the override input indication being received.

In aspect 27, the method of any of aspects 20-26 further includes that the model is based on modeling data comprising at least one of: endoscopic procedure information for a set of completed endoscopy procedures, anatomical information associated with one or more of the set of completed endoscopy procedures, patient information associated with one or more of the set of completed endoscopy procedures, endoscopy device information associated with one or more of the set of completed endoscopy procedures, endoscope information associated with one or more of the set of completed endoscopy procedures, or medical personnel information associated with one or more of the set of completed endoscopy procedures.

In aspect 28, the method of aspect 27 further includes that the model is an artificial intelligence (AI) / machine learning (ML) (AI/ML) model, wherein the modeling data is training data on which the AI/ML model is based.

In aspect 29, the method of aspect 27 or aspect 28 further includes that the endoscopic procedure information includes data associated with at least one of: an anticipated path of the endoscope, a first set of prior positional references associated with looping in a set of prior endoscopic procedures, or a second set of prior positional references associated with prior applications of pressure during the set of prior endoscopic procedures.

In aspect 30, the method of any of aspects 27-29 further includes that, for one or more endoscopy procedures in the set of completed endoscopy procedures on which the model is based, the anatomical information includes data associated with at least one of: positioning of a set of one or more anatomical features of a corresponding patient, size of the set of one or more anatomical features of the corresponding patient, shape of the set of one or more anatomical features of the corresponding patient, location of the set of one or more anatomical features of the corresponding patient, or a health assessment associated with the corresponding patient.

In aspect 31, the method of any of aspects 27-30 further includes that, for one or more endoscopy procedures in the set of completed endoscopy procedures on which the model is based, the patient information includes data associated with at least one of: a first orientation of a corresponding patient with respect to the endoscope, a second orientation of the corresponding patient with respect to a display for visual output associated with the endoscopic procedure, a set of patient dimensional measurements, or a medical history of the corresponding patient.

In aspect 32, the method of any of aspects 27-31 further includes that, for one or more endoscopy procedures in the set of completed endoscopy procedures on which the model is based, the endoscopy device information includes data associated with at least one of: a manufacturer of the endoscopy device used in a corresponding endoscopy procedure, a model type of the endoscopy device used in the corresponding endoscopy procedure, a set of adjustable pressure capabilities of the endoscopy device used in the corresponding endoscopy procedure, a set of visual output capabilities of the endoscopy device used in the corresponding endoscopy procedure, a set of sensor capabilities of the endoscopy device used in the corresponding endoscopy procedure, or a set of communication capabilities of the endoscopy device used in the corresponding endoscopy procedure.

In aspect 33, the method of any of aspects 27-32 further includes that, for one or more endoscopy procedures in the set of completed endoscopy procedures on which the model is based, the endoscope information includes data associated with at least one of: a manufacturer of the endoscope used in a corresponding endoscopy procedure, a model type of the endoscope used in the corresponding endoscopy procedure, a set of movement capabilities of the endoscope used in the corresponding endoscopy procedure, signal emitter information of the endoscope used in the corresponding endoscopy procedure, or a set of feedback information types of which the endoscope is configured to provide.

In aspect 34, the method of any of aspects 27-33 further includes that, for one or more endoscopy procedures in the set of completed endoscopy procedures on which the model is based, the medical personnel information includes data associated with a set of endoscopic procedure preferences of medical personnel, comprising at least one of: an amount of pressure applied for looping prevention in a corresponding endoscopy procedure, a set of patient orientations for the corresponding endoscopy procedure, a display of visual output for the corresponding endoscopy procedure, a manufacturer of the endoscope used in the corresponding endoscopy procedure, or a model type of the endoscope used in the corresponding endoscopy procedure.

In aspect 35, the method of any of aspects 20-34 further includes receiving at least one of outcome information or feedback of a medical result associated with the output of one or more of the flag identifying the potential loop, the recommendation of the action, or the instruction to adjust the external pressure device; and training or refining the model based on at least one of the outcome information or the feedback.

In aspect 36, the method of any of aspects 20-35 further includes receiving, as model input at least one of: endoscopic procedure information for the endoscopic procedure for the patient, anatomical information associated with the endoscopic procedure for the patient, patient information for the patient, endoscopy device information about the endoscopy device associated with the endoscopic procedure, endoscope information for the endoscope associated with the endoscopic procedure, or medical personnel information for one or more persons that will participate in the endoscopic procedure for the patient; and wherein model output from the model is an inference based on the model input.

In aspect 37, the method of any of aspects 20-36 further includes changing visual output, for at least one of a display or light emitting diode (LED) array associated with the endoscopy device, relative to a position of the endoscope and in accordance with the model.

In aspect 38, the method of aspect 37 further includes that the visual output comprises at least one of: a first indication of a presence of looping associated with the endoscopic procedure; a second indication of a looping prediction associated with the endoscopic procedure; a third indication of an absence of looping associated with the endoscopic procedure; or a fourth indication of a pressure position relative to the at least part of the abdomen of the patient corresponding to the presence of looping or the looping prediction for an application of the adjustable pressure.

Aspect 39 is a computer-readable medium storing computer executable code at a device, the code when executed by a processor causes the processor, utilizing a model to support an endoscopic procedure, to perform the method of any of aspects 20-38.

Aspect 40 is an apparatus utilizing a model to support an endoscopic procedure, the apparatus including means for performing the method of any of aspects 20-38.

What is claimed is:

1. An endoscopic procedure apparatus, comprising:
an input component configured to:
receive, from one or more sensors, information related to positioning of an endoscope in relation with a front portion of an endoscopy device or with a set of anatomical markers of a patient, wherein the endoscopy device is configured for placement around an abdomen of the patient during the endoscopic procedure and configured to apply an adjustable pressure, in accordance with a model, through compression on at least part of the abdomen of the patient; and
an output component configured to output, based at least in part on the information, one or more of:
a flag identifying an observed loop or predicting a potential loop at the patient during the endoscopic procedure,
a recommendation of an action that is predicted to avoid a first complication during the endoscopic procedure, or
instruction to adjust an external pressure device, wherein the instruction is for an adjustment that is predicted to avoid a second complication during the endoscopic procedure.

2. The apparatus of claim 1, further comprising:
a control unit configured to:
activate, for self-adjustment and in accordance with the instruction from the output component, an application of the adjustable pressure through the compression for the endoscopic procedure based on the information.

3. The apparatus of claim 2, wherein the control unit is configured to:
deactivate, for the self-adjustment and in accordance with the instruction from the output component, the application of the adjustable pressure through lessening of the compression for the endoscopic procedure based on the information.

4. The apparatus of claim 2, wherein the control unit is further configured to:
determine at least one of a presence of looping or a looping prediction during the endoscopic procedure based on the information and in accordance with the model;
wherein the control unit is configured to activate, for the self-adjustment and in accordance with the model, the application of the adjustable pressure through the compression for the endoscopic procedure based on the information and at a position relative to the at least part of the abdomen of the patient corresponding to the presence of looping or the looping prediction.

5. The apparatus of claim 2, wherein the control unit is configured to:
activate, for the self-adjustment and in accordance with the model, the application of the adjustable pressure at a specific level of pressure through the compression for the endoscopic procedure based on the information, or
wherein the control unit is configured to activate, for the self-adjustment and in accordance with the model, the application of the adjustable pressure through the compression for the endoscopic procedure based on the information and on endoscope feedback information.

6. The apparatus of claim 5, wherein the input component is further configured to:
receive the endoscope feedback information via a wired connection or a wireless connection with the endoscope; or
wherein the endoscope feedback information includes additional information associated with the endoscope, comprising at least one of orientation, flexion, or applied force.

7. The apparatus of claim 2, wherein the input component is further configured to:
receive an override input indication indicative of a termination of the adjustable pressure;
wherein the control unit is further configured to:
deactivate the application of the adjustable pressure based on the override input indication being received.

8. The apparatus of claim 1, wherein the model is based on modeling data comprising at least one of:
endoscopic procedure information for a set of completed endoscopy procedures,
anatomical information associated with one or more of the set of completed endoscopy procedures,
patient information associated with one or more of the set of completed endoscopy procedures,
endoscopy device information associated with one or more of the set of completed endoscopy procedures,
endoscope information associated with one or more of the set of completed endoscopy procedures, or
medical personnel information associated with one or more of the set of completed endoscopy procedures.

9. The apparatus of claim 8, wherein the model is an artificial intelligence (AI) / machine learning (ML) (AI/ML) model, wherein the modeling data is training data on which the AI/ML model is based.

10. The apparatus of claim 8, wherein the endoscopic procedure information includes data associated with at least one of:
an anticipated path of the endoscope,
a first set of prior positional references associated with looping in a set of prior endoscopic procedures, or
a second set of prior positional references associated with prior applications of pressure during the set of prior endoscopic procedures.

11. The apparatus of claim 8, wherein, for one or more endoscopy procedures in the set of completed endoscopy procedures on which the model is based, the anatomical information includes data associated with at least one of:
positioning of a set of one or more anatomical features of a corresponding patient,
size of the set of one or more anatomical features of the corresponding patient,
shape of the set of one or more anatomical features of the corresponding patient,
location of the set of one or more anatomical features of the corresponding patient, or
a health assessment associated with the corresponding patient.

12. The apparatus of claim 8, wherein, for one or more endoscopy procedures in the set of completed endoscopy procedures on which the model is based, the patient information includes data associated with at least one of:
a first orientation of a corresponding patient with respect to the endoscope,
a second orientation of the corresponding patient with respect to a display for visual output associated with the endoscopic procedure,
a set of patient dimensional measurements, or
a medical history of the corresponding patient.

13. The apparatus of claim 8, wherein, for one or more endoscopy procedures in the set of completed endoscopy procedures on which the model is based, the endoscopy device information includes data associated with at least one of:
a manufacturer of the endoscopy device used in a corresponding endoscopy procedure,
a model type of the endoscopy device used in the corresponding endoscopy procedure,
a set of adjustable pressure capabilities of the endoscopy device used in the corresponding endoscopy procedure,
a set of visual output capabilities of the endoscopy device used in the corresponding endoscopy procedure,
a set of sensor capabilities of the endoscopy device used in the corresponding endoscopy procedure, or
a set of communication capabilities of the endoscopy device used in the corresponding endoscopy procedure.

14. The apparatus of claim 8, wherein, for one or more endoscopy procedures in the set of completed endoscopy procedures on which the model is based, the endoscope information includes data associated with at least one of:
a manufacturer of the endoscope used in a corresponding endoscopy procedure,
a model type of the endoscope used in the corresponding endoscopy procedure,
a set of movement capabilities of the endoscope used in the corresponding endoscopy procedure,
signal emitter information of the endoscope used in the corresponding endoscopy procedure, or
a set of feedback information types of which the endoscope is configured to provide.

15. The apparatus of claim 8, wherein, for one or more endoscopy procedures in the set of completed endoscopy procedures on which the model is based, the medical personnel information includes data associated with a set of endoscopic procedure preferences of medical personnel, comprising at least one of:
an amount of pressure applied for looping prevention in a corresponding endoscopy procedure,
a set of patient orientations for the corresponding endoscopy procedure,
a display of visual output for the corresponding endoscopy procedure,
a manufacturer of the endoscope used in the corresponding endoscopy procedure, or
a model type of the endoscope used in the corresponding endoscopy procedure.

16. The apparatus of claim 1, further comprising a training component, wherein the training component is configured to:
receive at least one of outcome information or feedback of a medical result associated with the output of one or more of the flag identifying the potential loop, the recommendation of the action, or the instruction to adjust the external pressure device; and
train or refine the model based on at least one of the outcome information or the feedback.

17. The apparatus of claim 1, wherein the input component is further configured to receive, as model input at least one of:
endoscopic procedure information for the endoscopic procedure for the patient,
anatomical information associated with the endoscopic procedure for the patient,
patient information for the patient,
endoscopy device information about the endoscopy device associated with the endoscopic procedure,
endoscope information for the endoscope associated with the endoscopic procedure, or
medical personnel information for one or more persons that will participate in the endoscopic procedure for the patient; and
wherein model output from the model is an inference based on the model input.

18. The apparatus of claim 1, further comprising a visual indication component configured to:
change visual output, for at least one of a display or light emitting diode (LED) array associated with the endoscopy device, relative to a position of the endoscope and in accordance with the model.

19. The apparatus of claim 18, wherein the visual output comprises at least one of:
a first indication of a presence of looping associated with the endoscopic procedure;
a second indication of a looping prediction associated with the endoscopic procedure;
a third indication of an absence of looping associated with the endoscopic procedure; or
a fourth indication of a pressure position relative to the at least part of the abdomen of the patient corresponding to the presence of looping or the looping prediction for an application of the adjustable pressure.

20. A method utilizing a model to support an endoscopic procedure, comprising:
receiving, via an input component and from one or more sensors, information related to positioning of an endoscope in relation with a front portion of an endoscopy device or with a set of anatomical markers of a patient, wherein the endoscopy device is configured for placement around an abdomen of the patient during the endoscopic procedure and configured to apply an adjustable pressure, in accordance with the model, through compression on at least part of the abdomen of the patient; and
outputting, via an output component and based at least in part on the information, one or more of:
a flag identifying an observed loop or a potential loop that is predicted at the patient during the endoscopic procedure,
a recommendation of an action that is predicted to avoid a first complication during the endoscopic procedure, or
instruction to adjust an external pressure device, wherein the instruction is for an adjustment that is predicted to avoid a second complication during the endoscopic procedure.

21. A computer-readable medium storing computer executable code at a device, the code when executed by a processor causes the processor, utilizing a model to support an endoscopic procedure, to:
receive, via an input component and from one or more sensors, information related to positioning of an endoscope in relation with a front portion of an endoscopy device or with a set of anatomical markers of a patient, wherein the endoscopy device is configured for placement around an abdomen of the patient during the endoscopic procedure and configured to apply an adjustable pressure, in accordance with the model, through compression on at least part of the abdomen of the patient; and
output, via an output component and based at least in part on the information, one or more of:
a flag identifying a potential loop that is predicted at the patient during the endoscopic procedure,
a recommendation of an action that is predicted to avoid a first complication during the endoscopic procedure, or
instruction to adjust an external pressure device, wherein the instruction is for an adjustment that is predicted to avoid a second complication during the endoscopic procedure.
